(19) United States
(12) Patent Application Publication
(10) Pub. No.: US 2013/0324609 A1
(43) Pub. Date: Dec. 5, 2013

(54) DEPRESSION DISORDER THERAPEUTICS WITH CREATINE ANALOGS

(71) Applicants: Perry F. Renshaw, Salt Lake City, UT (US); Douglas G. Kondo, Salt Lake City, UT (US)

(72) Inventors: Perry F. Renshaw, Salt Lake City, UT (US); Douglas G. Kondo, Salt Lake City, UT (US)

(73) Assignee: UNIVERSITY OF UTAH RESEARCH FOUNDATION, Salt Lake City, UT (US)

(21) Appl. No.: 13/878,191

(22) PCT Filed: Oct. 7, 2011
(86) PCT No.: PCT/US11/55399

§ 371 Date: Jul. 23, 2013

Related U.S. Application Data
(60) Provisional application No. 61/391,542, filed on Oct. 8, 2010.

(51) Int. Cl. A61K 31/195; A61K 45/06

(52) U.S. Cl. CPC A61K 31/195; A61K 45/06
USPC 514/565

(57) ABSTRACT

In one aspect, the invention relates to creatine analogs, compositions comprising same, and methods of using same, alone or in combination with other agents, to treat depression disorders and associated maladies. This abstract is intended as a scanning tool for purposes of searching in the particular art and is not intended to be limiting of the present invention.

### CROSS-REFERENCE TO RELATED APPLICATIONS

This application claims the benefit of U.S. Application No. 61/391,542, filed Oct. 8, 2010, which is hereby incorporated by reference in its entirety.

### BACKGROUND

Adolescent major depressive disorder (MDD) is a public health problem associated with significant disability and mortality (Lewinsohn et al. 1998). Affecting up to 8% of U.S. adolescents at any given time (SAM HSA 2008), the lifetime prevalence of MDD prior to adulthood is estimated at up to 20% (Lewinsohn et al. 1993). Pediatric MDD is associated with negative outcomes including academic failure, social impairment, substance abuse and suicidal behavior (Birmaher et al. 2007; Fergusson and Woodward 2002). Mood disorders are the leading cause of hospitalization in the 13-17 age group (Owens et al. 2003), and MDD recurs: depressed adults are more likely to have had a depressive episode in adolescence (Fergusson et al. 2005), and youth MDD predicts depression and psychosocial impairment in adulthood (Pine et al. 1999). Thus, discovery of novel treatments for MDD in the critical adolescent stage of development has important public health implications (Fombonne et al. 2001b, a; Vitiello et al. 2006; Weissman et al. 1999).

The currently available treatments for adolescent MDD have significant limitations. Selective serotonin reuptake inhibitor (SSRI) antidepressants have comprised the principal pharmacologic treatment for adolescent MDD for more than a decade (Ma et al. 2005). However, at least 40% of adolescents receiving an SSRI fail to respond to treatment (Brent et al. 2008), and the response rate in rigorously conducted clinical trials favors antidepressants over placebo by just 11.0% (95% C.I. 7.1-14.9) (Usala et al. 2008). The concern has been raised that even this slim therapeutic margin is attributable to publication bias (Whittington et al. 2004). In addition to concerns regarding efficacy, meta-analyses and systematic reviews have suggested that antidepressants may increase the risk for suicidality (Hammad et al. 2006; Hetrick et al. 2007; Dubicka et al. 2006; Moller et al. 2008)—and attempted or completed suicide (Barbui et al. 2009)—when prescribed to children. Considered together, the severity of the illness and limitations of current therapy demonstrate the need for novel and more effective treatments for adolescent MDD.

Early childhood-onset mental illness (e.g., conduct problems, autism, ADHD) has a male preponderance, whereas adolescent-onset disorders (e.g., depression, anxiety) show a marked female preponderance (Zahn-Waxler et al. 2008). The prevalence of depression increases several-fold following puberty: from 1% in childhood to 8% in adolescence (Birmaher et al. 1996). A number of epidemiologic studies have shown that the burden of illness in this age group is borne primarily by females (Garrison et al. 1997; Hankin et al. 1998; Wichstrom 1999; Wade et al. 2002).

Therefore, there remains a need for methods and compositions that overcome these deficiencies and that effectively treatment of depression in humans. A further need is improved treatment of depression in adolescent humans, especially female adolescent humans.

### SUMMARY

In accordance with the purpose(s) of the invention, as embodied and broadly described herein, the invention, in one aspect, relates to a creatine analog for the treatment of a depression disorder, pharmaceutical compositions comprising same, and methods of treating same.

Disclosed are methods for the treatment of a mammal diagnosed with a depression disorder comprising the step of administering to the mammal an effective amount of at least one creatine analog.

Also disclosed are methods for reducing risk of suicide in a patient having a depression disorder comprising the step of administering to the patient an effective amount of at least one creatine analog.

Also disclosed are methods of reducing likelihood of depression symptoms in a subject comprising the step of administering to the patient an effective amount of at least one creatine analog within ten days of administration to the subject an agent known to have a side effect of causing depression.

Also disclosed are methods for the treatment of a depression disorder in a selective serotonin reuptake inhibitor-treatment resistant patient comprising the step of administering to the mammal an effective amount of a selective serotonin reuptake inhibitor and an effective amount of at least one creatine analog.

Also disclosed are methods for the treatment of a subject comprising the steps of diagnosing the subject as having a depression disorder; and administering to the subject an effective amount of at least one creatine analog.

Also disclosed are oral dosage forms comprising at least one creatine analog and one or more of at least one agent known to treat a depression disorder; or at least one agent known to have a side effect of causing depression.

Also disclosed are kits comprising at least one creatine analog and one or more of at least one agent known to treat a depression disorder; at least one agent known to have a side effect of causing depression; or instructions for treating a disorder associated with depression.

Also disclosed are pharmaceutical compositions comprising a therapeutically effective amount of a disclosed compound and a pharmaceutically acceptable carrier.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative CDRS-R Scores during treatment with adjunctive creatine.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples and Figures included herein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound If given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more neurological and/or psychiatric disorder associated depression disorder prior to the administering step. In some aspects of the disclosed method, the subject has been diagnosed with an increased risk of suicide or suicidal ideation prior to the administering step. In some aspects of the disclosed method, the subject is receiving a therapeutic agent associated with an increased risk of suicide or suicidal ideation prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a clinical interview and/or a physical examination by a person of skill, for example, a physician, and found to have a condition that can be diagnosed or treated by the compounds, compositions, or methods disclosed herein. For example, “diagnosed with a depression disorder” means having been subjected to a clinical interview and/or physical examination by a person of skill, for example, a physician, and found to have a condition that can be diagnosed or treated by a compound or composition that can cure, alleviate, prevent, or otherwise treat a depression disorder.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to depression) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target, either directly; i.e., by interacting with the target itself, or indirectly; i.e., by interacting with another molecule, co-factor, factor, or protein on which the activity of the target is dependent.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The term “stable,” as used herein, refers to compounds that are not substantially altered when subjected to conditions to allow for their production, detection, and, in certain aspects, their recovery, purification, and use for one or more of the purposes disclosed herein.

Compounds described herein comprise atoms in both their natural isotopic abundance and in non-natural abundance. The disclosed compounds can be isotopically-labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, such as ²H, ³H, ¹³C, ¹⁴C, ¹⁵N, ¹⁸O, ¹⁷O, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. CREATINE ANALOGS

In one aspect, the invention relates to creatine analogs, or pharmaceutically acceptable salts thereof, having a structure represented by a formula:

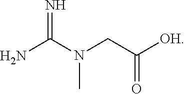

In one aspect, creatine analogs comprise creatine, creatine salts, creatine esters, creatine amides and creatine hydrates. In a further aspect, creatine esters comprise alkyl esters. In a yet further aspect, creatine esters comprise creatine ethyl ester. In a further aspect, creatine hydrates comprise creatine monohydrate. In a further aspect, creatine salts comprise the carboxylate anion form of creatine and a pharmaceutically acceptable cation counterion. In a still further aspect, creatine salts comprise protonation of the primay amine of creatine with an acid.

In one aspect, creatine analogs comprise one or more of creatine, creatine monohydrate, creatine ethyl ester, creatine citrate, creatine malate, creatine tartrate, and magnesium creatine chelate. In a further aspect, creatine analogs comprise creatine monohydrate. In a still further aspect, creatine analogs comprise creatine ethyl ester. In a yet further aspect, creatine analogs comprise magnesium creatine chelate. In a further aspect, creatine analogs comprise creatine monohydrate, creatine ethyl ester and magnesium creatine chelate.

In a further aspect, creatine analogs comprise one or more compounds having a structure of:

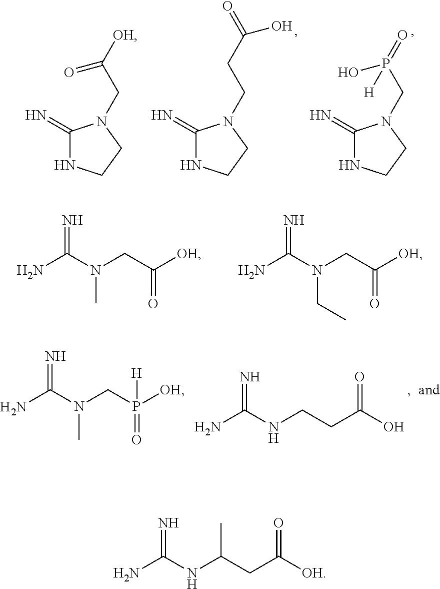

In one aspect, the creatine analog is a prodrug form of creatine, wherein prodrug is an analogue which upon administration to the recipient is capable of providing (directly or indirectly) the compound, or an active metabolite or residue thereof. Such prodrugs are recognizable to those skilled in the art, without undue experimentation. Nevertheless, reference is made to the teaching of Burger's Medicinal Chemistry and Drug Discovery, 5 th Edition, Vol 1: Principles and Practice, which is incorporated herein by reference to the extent of teaching such derivatives.

In a further aspect, a prodrug of a creatine analog of the invention is converted within the body, e.g. by hydrolysis in the blood, into its active form that has medical effects. Pharmaceutically acceptable prodrugs are-described in T. Higuchi and V. Stella, Prodrugs as Novel Delivery Systems, Vol. 14 of the A.C.S. Symposium Series, Edward B. Roche, ed.; Bioreversible Carriers in Drug Design, American Pharmaceutical Association and Pergamon Press, 1987; and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

In a further aspect, a prodrug of a creatine analog of the invention are any covalently bonded carriers that release the compound in vivo when such prodrug is administered to a patient. In a still further aspect, a prodrug is prepared by modifying a functional group in a way such that the modification is cleaved, either by routine manipulation or in vivo, yielding the parent compound. In a still further aspect, a prodrug includes, for example, compounds wherein the amine group is bonded to any group that, when administered to a patient, cleaves to form the amine group. Thus, representative examples of prodrugs include (but are not limited to) acetate, formate and benzoate derivatives of the amine functional group.

When used herein, the term “alkyl” refers to straight and branched groups containing up to six carbon atoms. Examples of such groups include methyl, ethyl, propyl, isopropyl, n-butyl, isobutyl, tert-butyl, pentyl or hexyl.

When used herein, the term “aryl” refers to, unless otherwise defined, single or fused aromatic rings suitably containing from 4 to 7, preferably 5 or 6, ring atoms in each ring. A fused ring system may include aliphatic rings and need only include one aromatic ring. Examples of suitable aryl rings include phenyl and naphthyl.

When used herein, the term “alkanol” refers to C 1-9 alkyl alcohols, for example methanol, ethanol, industrially methylated spirit (IMS), n-propanol, iso-propanol (IPA), n-butanol, pentanol, hexanol, heptanol, octanol or nonanol, in particular methanol, ethanol, IMS, IPA or n-butanol.

In one aspect, the creatine analog is a pharmaceutically acceptable derivative. In a further aspect, the pharmaceutically acceptable derivative is a salt, solvate, ester, carbamate and phosphate ester. In a still further aspect, “derivative” means any pharmaceutically acceptable derivative or non-pharmaceutically acceptable derivative which is suitable for use in the process of the present invention. The skilled person will appreciate that non-pharmaceutically acceptable derivatives may be used to prepare compounds and derivatives suitable for pharmaceutical use.

In one aspect, creatine analogs comprise compounds prepared as a pharmaceutically acceptable salt. For a review on suitable salts see Berge et al., J. Pharm. Sci., 1977, 66, 1-19. In a still further aspect, a pharmaceutically acceptable salt may be readily prepared by using a desired acid or base as appropriate. The salt may precipitate from solution and can be collected by filtration or may be recovered by evaporation of the solvent. In a yet further aspect, salts comprise acid addition salts resulting from reaction of an acid with a basic nitrogen atom.

In a further aspect, a salt within the term “pharmaceutically acceptable salts” refer to non-toxic salts of the creatine analogs of the invention. In a still further aspect, addition salts are formed from acids which form non-toxic salts and comprise acetate, p-aminobenzoate, ascorbate, aspartate, benzenesulfonate, benzoate, bicarbonate, bismethylenesalicylate, bisulfate, bitartrate, borate, bromide, calcium edetate, camsylate, carbonate, chloride, clavulanate, citrate, cyclohexylsulfamate, dihydrochloride, edetate, edisylate, estolate, esylate, ethanedisulfonate, ethanesulfonate, formate, fumarate, gluceptate, gluconate, glutamate, glutarate, glycollate, glycollylarsanilate, hemisulfate, hexylresorcinate, hydrabamine, hydrobromide, hydrochloride, hydrogen phosphate, hydroiodide, hydroxynaphthoate, iodide, isethionate, itaconate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, oxaloacetate, pamoate (embonate), palmate, palmitate, pantothenate, phosphate/diphosphate, piruvate, polygalacturonate, propionate, saccharate, salicylate, stearate, subacetate, succinate, sulfate, tannate, tartrate, teoclate, tosylate, triethiodide, trifluoroacetate and valerate. Preferred salts prepared according to the present invention include the succinate, glutarate and hemisulfate salts.

In one aspect, the invention relates to creatine analogs or pharmaceutically acceptable salts thereof having a structure represented by a formula:

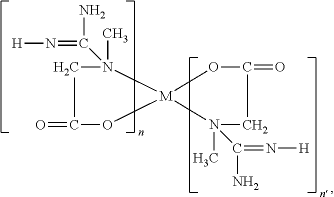

wherein M is a metal, n is 1 and n′ is 0, 1, or 2. In a further aspect, n′ is 0 providing a ligand to metal molar ratio of 1:1. For example, magnesium creatine may have a ligand to metal molar ratio of 2:1 (n′=1). In a yet further aspect, the metal molar ratio is 1:1 (n′=0). In a still further aspect, ligand to metal molar ratios can be creatine to calcium at 1:1 (n′=0); creatine to zinc at 1:1 (n′=0); creatine to chromium at 1:1 (n′=0), 2:1 (n′=1) and/or 3:1 (n′=2); creatine to manganese at 1:1 (n′=0); and creatine to iron a 1:1 (n′=0), 2:1 (n′=1) and/or 3:1 (n′=2). When n′=0, one or more anions can be present in the solution in a structure represented by a formula:

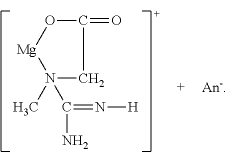

The bonds depicted between the metal (M) and the amine group and between the metal (M) and carboxyl oxygen group as shown and described should not necessarily be strictly construed to represent coordinate covalent bonds. For example, in one aspect, a covalent bond may exists between the metal (M) and the amine group whereas an ionic or coulombic bond exists between the metal (M) and the carboxyl oxygen group in a structure represented by a formula:

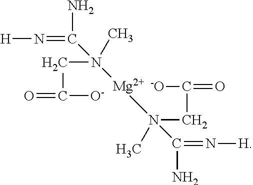

In a further aspect, the net electrical charge at the metal ion is preferably zero. For example, positive charge on the metal ion can be neutralized by electrons contributed by the ligand in formation of the heterocyclic chelate ring.

A method of preparing the creatine chelates of the present invention is as follows. First, a soluble metal salt or an insoluble metal compound is dissolved in water or solubilized in an acidic solution respectively. If an acidic solution is required to disassociate the metal ions, acids such as acetic, citric, lactic, malic, hydrochloric, phosphoric, sulfuric, tartaric, maleic and naturally occurring amino acids such as aminobutyric, aspartic and glutamic acids, etc., may be used. If a metal salt is used that is soluble in water, it may not be required to use an acidic solution, though it may be desired. For example, if magnesium is the metal to be chelated, magnesium sulfate, magnesium citrate, magnesium chloride, magnesium phosphate monobasic, magnesium nitrate, magnesium oxide, etc., may be used as the metal source which will either be dissolved in water or acidified in an acidic solution. To this solution, a creatine ligand is then added. If the pH level is not around neutral, i.e., if it is between about 7.5 and 10, a pH adjuster may be added. pH adjusters may include o-phosphoric acid, citric acid, malic acid, acetic acid, hydrochloric acid, tartaric acid, lactic acid, nitric acid, sulfuric acid and naturally occurring amino acids such as aminobutyric acid, aspartic acid and glutamic acid among others, though o-phosphoric acid is preferred. For example if a creatine chelate is prepared by reacting a creatine ligand with a metal oxide in the presence of citric acid, o-phosphoric acid or another acidifying agent may be added to lower the pH from more basic levels (about 7.5 to 10) to a more neutral pH (about 7).

In a further aspect, the order that one mixes the ingredients is not central to the method of preparing creatine chelates. The creatine ligand can be added to the aqueous acidic solution first followed by the addition of the metal, or even simultaneously

In one aspect, magnesium creatine, can be prepared by reacting magnesium oxide, creatine, o-phosphoric acid and citric acid in an aqueous environment. The formulation is stoichiometrically balanced so that no unreacted magnesium oxide remains in the product. Of the possible combinations and permutations, one structure is provided above. In a further aspect, the ligand to metal molar ratio is 1:1 and the anion may be any of a number of possible corresponding anions such as chloride (Cl⁻), iodide (I⁻), bisulfate (HSO₄⁻), bicarbonate (HCO₃⁻), dihydrogen phosphate (H2PO₄⁻), phosphate (PO₄⁻), sulfate (SO₄²⁻), citrate, acetate (C₂H₃O₂⁻), lactate, malate, aminobutyrate, aspartate and glutamate or anions from other soluble salts. In a yet further aspect, the ligand to metal molar ratio is more than 1:1, wherein another creatinate anion is present.

In a further aspect, each disclosed derivative can be optionally further substituted. In a yet further aspect, any one or more derivative can be optionally omitted from the invention. It is understood that a disclosed compound can be provided by the disclosed methods. It is also understood that the disclosed compounds can be employed in the disclosed methods of using.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

In one aspect, the invention relates to compounds, or pharmaceutically acceptable salts thereof, having a structure represented by a formula:

### C. DIAGNOSIS OF DEPRESSION DISORDER

In one aspect, a method of diagnosis of a depression disorder comprises determining, in a brain of a patient, levels of a marker (e.g., a metabolite) indicative of a brain bioenergetic metabolic state of the patient, the brain bioenergetic metabolic state being predictive as to whether the patient will manifest reduced symptoms of depression in response to a depression treatment. In a still further aspect, the marker is detected in a region of the brain comprising at least one of the anterior cingulate, the amygdala, and the hippocampus of the brain. In one aspect, the method of diagnosis of depression comprises a mood disorder, such as depression or bipolar disorder. major depressive disorder. In a further aspect, the patient is under about 25 years of age. In an even further aspect, the patient has suicidal ideation. In a still further aspect, the patient is an adolescent. In an even further aspect, the patient is about 13 to about 18 years of age. In a further aspect, the patient is an adolescent that is resistant to treatment with a selective serotonin reuptake inhibitor. In a still further aspect, the adolescent is female.

In one aspect, the marker comprises at least one of adenosine triphosphate, adenosine diphosphate, and phosphocreatine. In a further aspect, the first and second levels of the marker are determined by ³¹P magnetic resonance spectroscopy or MR spectroscopy of another suitable isotope. In a still further aspect, the marker can comprise at least one of magnesium, pH, total nucleoside triphosphate, and β NTP. In a yet further aspect, the marker can also be any other known to those of skill in the art. In an even further aspect, the marker comprises phosphocreatine.

In one aspect, a marker comprises a brain bioenergetic metabolic state marker, wherein the brain energetic metabolic state marker comprises a pH, a compound comprising magnesium, and a compound comprising phosphorus (e.g., PCr, ATP, ADP, Pᵢ, total NTP, α-NTP, β-NTP, γ-NTP, and combinations thereof). As used herein, “PCr” means phosphocreatine. In a further aspect, levels of such phosphorus comprising compounds present in the brain of a patient can be determined by, for instance, ³¹P MRS. In a still further aspect, the patient can suffer from major depression disorder. In a further aspect, the patient can suffer from depression resulting from recurring head pain, such as migraine headaches, cluster headaches, and tension headaches. In a still further aspect, the antidepression treatment can comprise administering to the patient an SSRI, a tricyclic, a thyroid hormone, or combinations thereof.

In one aspect, brain levels of ADP, ATP, and PCr are different, as compared to a subject that does not suffer from depression, in the brain of a subject that suffers from depression and that will likely manifest reduced levels and/or symptoms of depression in response to an antidepression treatment. In some embodiments, an antidepression treatment results in a substantial normalization of brain levels of ADP, ATP, and PCr in the brain of a patient that manifests reduced levels and/or symptoms of depression in response to the antidepression treatment. In a further aspect, normalizing changes in brain PCr and ATP levels in can result in the achievement of a substantially normalized brain bioenergetic metabolic state as a result of the buffer role of PCr in relation to ATP. For example, brain concentrations of ATP can, at the expense of brain PCr concentrations, normally be maintained at substantially uniform levels by PCr transfer of a high-energy phosphate group to ADP, re-forming ATP in a reaction mediated by, for example, creatine kinase. A reduction of an brain concentration of ADP, ATP, or PCr to a substantially non-physiologic level can result in a brain metabolic state correlated with depression. An antidepression treatment that substantially normalizes a level of ADP, ATP, or PCr in a patient suffering from depression can thereby alleviate a level or symptom of depression in the patient. But such normalizing changes in brain ADP, ATP, and PCr brain concentrations in patients that respond to a depression treatment can also be achieved by other mechanisms.

In one aspect, a mitochondrial dysfunction characterizes a patient that manifests reduced levels and/or symptoms of depression in response to an antidepression treatment modality. In a further aspect, low levels of magnesium in the brain of a subject that suffers from depression, as compared with normal subjects, can result from impaired oxidative phosphorylation related to mitochondrial dysfunction; and impaired oxidative phosphorylation can result in a brain bioenergetic metabolic state correlated with depression. An antidepression treatment that substantially normalizes, in a patient suffering from depression, brain magnesium levels resulting from mitochondrial dysfunction can alleviate a level or symptom of depression in the patient. But such normalizing changes in levels of magnesium in the brain of a patient that responds to a depression treatment modality can also be achieved by other mechanisms.

In one aspect, diagnosis of a depression disorder comprises a medical history. In a further aspect, symptoms of depression can include, for example, depressive mood, hypobulia, loss of interest and pleasure, disrupted concentration and attention, lowered self-esteem and self-confidence, feelings of guilt and worthlessness, pessimism about the future, thoughts of suicide, sleep disorders, and loss of appetite. These symptoms have features peculiar to depression, which differ from depressed feelings experienced by anyone, and also differ from the lowered mental activity and sense of exhaustion experienced by people afflicted with physical diseases. The symptoms of depression are mainly comprehended by taking a precise medical history, questioning when and how the symptoms in terms of mental activity were developed and what types of damages have been imposed upon their social and domestic lives, and confirming various symptoms based on a patient's attitude or the contents of conversations during consultation. For example, family medical history, anamnesis, physical conditions, early developmental history, life history, personality inclination, premorbid social adaptation, and the occurrence of any episode(s) that had triggered the disease can be important references. In order to accurately comprehend these factors, an interview needs to be conducted by a highly skilled specialist in psychiatric medicine for approximately 1 hour. Further, it should be confirmed that a patient does not have any major abnormalities in terms of general physical or neurological conditions. If necessary, the possibility of the existence of organic brain disorders is to be eliminated by electroencephalography or brain imaging tests. The patient is then subjected to diagnosis.

In one aspect, the diagnosis comprises comparing the findings of the medical history with the diagnostic standards. Diagnostic standards comprise those provided in DSM-IV: Diagnostic and Statistical Manual of Mental Disorders—Fourth Edition, Text Revision (American Psychiatric Association, 2000), including, but not limited to, revisions, updates and new editions. In a further aspect, the diagnostic standards provided in the International Statistical Classification of Diseases and Related Health Problems 10th Revision (ICD-10) published by the World Health Organization, including, but not limited to, revisions, updates and new editions.

In one aspect, a medical practioner, including, but not limited to, a psychiatrist, medical doctor, psychologist, licensed social worker, nurse, physician assistant, professional counselor, or substance abuse counselor, can use a “depression symptoms rating scale”. The term “depressions symptoms rating scale” means any one of a number of standardized questionnaires, clinical instruments, or symptom inventories utilized to measure symptoms and symptom severity in depression. Such rating scales are often used in clinical practice to assess a subject or assist in providing a diagnosis. Such rating scales are often used in clinical studies to define treatment outcomes, based on changes from the study's entry point(s) to endpoint(s). In further aspect, a depression symptoms rating scale comprises one or more of Apathy Scale of Glenn et al., Bech-Rafaelsen Melancholia Scale, Beck Depression Inventory (BDI), Beck Depression Inventory II (BDI-II), Brief Screening Instrument of Fabacher et al to Detect Depression in an Elderly Patient in the Emergency Department (ED-DSI), Burns Depression Checklist (BDC), Center for Epidemiologic Studies Depression Scale—Revised (CESD-R), Center for Epidemiologic Studies Depression Scale (CES-D), Center for Epidemiological Studies Depression Scale for Children (CES-DC), Children's Depression Inventory (CDI), Children's Depression Rating Scale, Revised (CDRS-R), Clinical Global Impression Scale-I, Clinician Administered Posttraumatic Stress Disorder (PTSD) Scale-2 (CAPS), Cornell Scale for Depression in Dementia (CSDD), Depression and Anxiety in Youth Scale (DAYS), Depression Anxiety Stress Scales (DASS), Depression Outcomes Module (DOM), Diagnostic and Statistical Manual of Mental Disorders 4th Edition (DSM IV), Edinburgh Postnatal Depression Scale (EPDS), Geriatric Depression Scale (GDS; long or short format), Global Assessment of Functioning Scale, Goldberg Depression & Mania Scales, Hamilton Anxiety Rating Scale, Hamilton Depression Rating Scale (HDRS), Hamilton Depression Scale (HAM-D), Harvard National Depression Screening Scale (HANDS), Hospital Anxiety and Depression Scale (HADS), International Statistical Classification of Diseases and Related Health Problems 10th Revision (ICD-10), K-SADS Depression Rating Scale (K-DEP), KSADS Lifetime and Present (KSADS-PL) schedule, Liebowitz Social Anxiety Scale, Major Depression Inventory (MDI), Medical Outcomes Study Depression Questionnaire, Mehrabian Trait Anxiety and Depression Scales, Mini-Mental State Examination, Montgomery-Asberg Depression Rating Scale, Multiscore Depression Inventory (MDI), Multiscore Depression Inventory for Children (MDI-C), Newcastle Diagnostic and ECT Scales of Carney et al for Depression, Online Depression Screening Test (ODST), Panic Disorder Severity Scale, Postpartum Depression Screening Scale (PDSS), Post-Stroke Depression Rating Scale of Gainotti et al, RAND Self Administered Depression Screener, Raskin Scale or Three-Area Severity of Depression Scale, Revised Hamilton Rating Scale for Depression (RHRSD), Reynolds Adolescent Depression Scale (RADS), Reynolds Adolescent Depression Scale, Second Edition (RADS-2), Reynolds Child Depression Scale (RCDS), Risk Factors of Beck for Postpartum Depression (PPD), Risk Factors of Kivela et al for Predicting Chronic Depression in the Elderly, Sheehan Disability Scale, Treatment Outcome PTSD Scale, Yale-Brown Obsessive Compulsive Scale, and Zung Self-Rated Depression Scale. In a yet further aspect, the depression symptoms rating scale comprises a new edition, revision or update to one of the above depression symptoms rating scales.

In one aspect, a medical practioner, including, but not limited to, a psychiatrist, medical doctor, psychologist, licensed social worker, nurse, physician assistant, professional counselor, or substance abuse counselor, can use a “suicide symptoms rating scale”. The term “suicide symptoms rating scale” means any one of a number of standardized questionnaires, clinical instruments, or symptom inventories utilized to measure symptoms and symptom severity in depression. Such rating scales are often used in clinical practice to assess a subject or assist in providing a diagnosis. Such rating scales are often used in clinical studies to define treatment outcomes, based on changes from the study's entry point(s) to endpoint(s). In further aspect, a suicide symptoms rating scale comprises one or more of Adolescent Inventory of Suicide Orientation-30 (ISO-30), Adult Suicidal Ideation Questionnaire (ASIQ), Beck Hopelessness Scale (BHS), Beck Scale for Suicide Ideation (BSS), Child Suicide Risk Assessment (CSRA), Child-Adolescent Suicidal Potential Index (CASPI), Columbia Classification Algorithm of Suicide Assessment (C-CASA), Columbia Suicide Severity Rating Scale (C-SSRS), Coping Inventory for Stressful Situations (CISS), Firestone Assessment of Self-Destructive Thoughts (FAST), Lazurus' BASIC ID tool, Lifetime Parasuicide Count (LPC), MAST—Attraction to Death (MAST-AD), MAST—Repulsion by Life (MAST-RL), Modified SAD PERSONS Scale of Hockberger and Rothstein, Multi-Attitude Suicide Tendency Scale (MAST), Parasuicide History Interview (PHI), Positive and Negative Suicide Ideation Inventory (PANSI), Reasons for Living Inventory (RFL; either long form or short form), Reasons for Living Inventory for Adolescents (RFL-A), Reasons for Living Inventory for Young Adults (RFL-YA), Risk Factors of Powell et al for Predicting the Risk of Suicide in a Psychiatric Ward Inpatient, Risk-Rescue Rating (of Weisman and Worden for Suicide Assessment), Scale for Suicidal Ideation (SSI), Suicidal Behavior History Form (SBHF), Suicidal Behavior Questionnaire for Children (SBQ-C), Suicidal Ideation Questionnaire (SIQ), Suicidal Tendencies Test, Suicide Behaviors Questionnaire (SBQ), Suicide Behaviors Questionnaire-Revised (SBQ-R), Suicide Probability Scale (SPS), and Suicide Resilience Inventory-25 (SRI-25). In a still further aspect, a suicide symptom rating scale comprises a revision, new edition, or derivation of one of a suicide symtptom rating scale. In a yet further aspect, the suicide symptoms rating scale comprises a new edition, revision or update to one of the above suicide symptoms rating scales.

In one aspect, a medical practioner, including, but not limited to, a psychiatrist, medical doctor, psychologist, licensed social worker, nurse, physician assistant, professional counselor, or substance abuse counselor, has determined that a patient has Children's Depression Rating Scale-Revised (CDRS-R) raw score>40. In a yet further aspect, the patient with a Children's Depression Rating Scale-Revised (CDRS-R) raw score>40 has been treated with a selective serotonin reuptake inhibitor for ≧8 weeks. In a still further aspect, the selective serotonin reuptake inhibitor is fluoxetine. In an even further aspect, the patient has suicidal ideation. In a further aspect, the patient is under about 25 years of age. In a still further aspect, the patient is an adolescent. In an even further aspect, the patient is about 13 to about 18 years of age. In a further aspect, the patient is an adolescent that is resistant to treatment with a selective serotonin reuptake inhibitor. In a still further aspect, the adolescent is female.

In one aspect, ³¹P-MRS spectra are acquired from the patient to determine PCr levels. In a still further aspect, the ³¹P-MRS spectra show an increase in PCr levels in the patient upon treatment with a creatine analog. In an even further aspect, the increase in PCr levels in the patient is detected after about 1-4 weeks of treatment with a creatine analog. In a further aspect, the increase in PCr levels in the patient is maintained for at least one week upon discontinuation of treatment with a creatine analog. In a still further aspect, the PCr levels continues to increase for at least one week in the patient upon discontinuation of treatment with a creatine analog. In an even further aspect, there is no change significant change in β-NTP levels, pH or PCr/β-NTP ratio in the patient upon treatment with a creatine analog. In a further aspect, there is no significant difference in the patient in the β-NTP levels, pH or PCr/β-NTP ratio compared to a normal subject. In an even further aspect, the patient has suicidal ideation. In a further aspect, the patient is under about 25 years of age. In a still further aspect, the patient is an adolescent. In an even further aspect, the patient is about 13 to about 18 years of age. In a further aspect, the patient is an adolescent that is resistant to treatment with a selective serotonin reuptake inhibitor. In a still further aspect, the adolescent is female.

In one aspect, ³¹P-MRS spectra are acquired from a patient to determine one or more of β-NTP levels, total phosphorus levels, phosphomonoester levels, phosphodiester levels, phosphocholine levels, and pH, In a further aspect, a patient's CDRS-R score is positively correlated with baseline pH. In a still further aspect, a patient's a patient's CDRS-R score is negatively correlated with β-NTP concentration. In a yet further aspect, a patient's pre-treatment β-NTP concentration is not lower than a non-depressed patient. In an even further aspect, the patient's β-NTP levels, pH or PCr/b-NTP ratio do not change with treatment using a creatine analog. In an even further aspect, the patient has suicidal ideation. In a further aspect, the patient is under about 25 years of age. In a still further aspect, the patient is an adolescent. In an even further aspect, the patient is about 13 to about 18 years of age. In a further aspect, the patient is an adolescent that is resistant to treatment with a selective serotonin reuptake inhibitor. In a still further aspect, the adolescent is female.

### D. PHARMACEUTICAL COMPOSITIONS

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In certain aspects, the disclosed pharmaceutical compositions comprise the disclosed compounds and pharmaceutically acceptable salt(s) thereof as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carriers) followed by chilling and shaping in moulds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions which require modulation of symptoms associated with depression disorder the appropriate dosage level will generally be about 0.5 to 50 g/day of a creatine analog per patient and can be administered in a single dose or multiple doses. Preferably the dosage level will be about 1 to 20 g/day per patient. In a further aspect, the effective amount is from about 1 to about 20 g/day of a creatine analog per patient, more preferably about 2 to 15 g/day of a creatine analog per patient. A suitable dosage level of a creatine analog can be about 1 to about 10 g/day, from about 1 to about 8 g/day, from about 1 to about 5 g/day, from about 2 to about 10 g/day, from about 2 to about 8 g/day, from about 2 to about 5 g/day, from about 3 to about 10 g/day, from about 3 to about 8 g/day, or from about 3 to about 5 g/day. In a further aspect, the effective amount is about 1 g/day, about 2 g/day, about 3 g/day, about 4 g/day, about 5 g/day, about 6 g/day, about 7 g/day, about 8 g/day, about 9 g/day, or about 10 g/day. The compound can be administered on a regiment of 1 to 4 times per day, preferably once or twice per day. This dosing regiment can be adjusted to provide the optimal therapeutic response.

In the treatment conditions which require modulation of symptoms associated with suicidal ideation the appropriate dosage level will generally be about 0.5 to 50 g/day of a creatine analog per patient and can be administered in a single dose or multiple doses. Preferably the dosage level will be about 1 to 20 g/day per patient. In a further aspect, the effective amount is from about 1 to about 20 g/day of a creatine analog per patient, more preferably about 2 to 15 g/day of a creatine analog per patient. A suitable dosage level of a creatine analog can be about 1 to about 10 g/day, from about 1 to about 8 g/day, from about 1 to about 5 g/day, from about 2 to about 10 g/day, from about 2 to about 8 g/day, from about 2 to about 5 g/day, from about 3 to about 10 g/day, from about 3 to about 8 g/day, or from about 3 to about 5 g/day. In a further aspect, the effective amount is about 1 g/day, about 2 g/day, about 3 g/day, about 4 g/day, about 5 g/day, about 6 g/day, about 7 g/day, about 8 g/day, about 9 g/day, or about 10 g/day. The compound can be administered on a regiment of 1 to 4 times per day, preferably once or twice per day. This dosing regiment can be adjusted to provide the optimal therapeutic response.

In the treatment conditions wherein the patient is receiving a therapeutic agent associated with an increased risk of suicide or suicidal ideation the dose the appropriate dosage level will generally be about 0.5 to 50 g/day of a creatine analog per patient and can be administered in a single dose or multiple doses. Preferably the dosage level will be about 1 to 20 g/day per patient. In a further aspect, the effective amount of a creatine analog is from about 1 to about 20 g/day of a creatine analog per patient, more preferably about 2 to 15 g/day of a creatine analog per patient. A suitable dosage level can be about 1 to about 10 g/day, from about 1 to about 8 g/day, from about 1 to about 5 g/day, from about 2 to about 10 g/day, from about 2 to about 8 g/day, from about 2 to about 5 g/day, from about 3 to about 10 g/day, from about 3 to about 8 g/day, or from about 3 to about 5 g/day. In a further aspect, the effective amount is about 1 g/day, about 2 g/day, about 3 g/day, about 4 g/day, about 5 g/day, about 6 g/day, about 7 g/day, about 8 g/day, about 9 g/day, or about 10 g/day. The compound can be administered on a regiment of 1 to 4 times per day, preferably once or twice per day. This dosing regiment can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The present invention is further directed to a method for the manufacture of a medicament for modulating glutamate receptor activity (e.g., treatment of one or more neurological and/or psychiatric disorder associated with glutamate dysfunction) in mammals (e.g., humans) comprising combining one or more disclosed compounds, products, or compositions with a pharmaceutically acceptable carrier or diluent. Thus, in one aspect, the invention relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

### E. METHODS OF USING THE COMPOUNDS AND COMPOSITIONS

The compounds disclosed herein are useful for treating, preventing, ameliorating, controlling or reducing the risk of a variety of depression disorders. Thus, provided is a method of treating or preventing a disorder in a subject comprising the step of administering to the subject at least one disclosed compound; at least one disclosed pharmaceutical composition; and/or at least one disclosed product in a dosage and amount effective to treat the disorder in the subject.

The pharmaceutical compositions and methods of the present invention can further comprise other therapeutically active compounds as noted herein which are usually applied in the treatment of the above mentioned pathological conditions.

In various aspects, the disclosed treatment methods can be applied to a subject, for example, a patient. In further aspects, the subject is a mammal, for example, a human. In further aspects, the subject is an adolescent. In further aspects, the subject is a female. In a yet further aspect, the subject is selected from a geriatric patient, an individual with mild to traumatic brain injury, a military veteran with a history of post-traumatic stress disorder, a military veteran with combat experience, a military veteran with mild to severe traumatic brain injury, a individual less than about 25 years of age who is prescribed an anti-depressant therapeutic agent, and an individual who has a history of substance abuse. In an even further aspect, the patient has suicidal ideation. In a still further aspect, the patient is an adolescent. In an even further aspect, the patient is about 13 to about 18 years of age. In a further aspect, the patient is an adolescent that is resistant to treatment with a selective serotonin reuptake inhibitor. In a still further aspect, the adolescent is female.

In a further aspect, the subject is selected from a military veteran with combat experience, a military veteran with a history of post-traumatic stress disorder, and a military veteran with mild to severe traumatic brain injury. In a still further aspect, the subject is a military veteran with mild traumatic brain injury. In a yet further aspect, the subject is a military veteran with severe traumatic brain injury. In an even further aspect, a the subject is a military veteran with mild to severe traumatic brain injury. In a further aspect, the subject is a military veteran with combat experience. In a yet further aspect, In a further aspect, the subject is a military veteran with a history of post-traumatic stress disorder.

In a further aspect, the subject is a geriatric patient. In a still further aspect, the subject is a geriatric patient who is prescribed an anti-depressant therapeutic agent. In a yet further aspect, the geriatric patient is in about the first 90 days of treatment with an anti-depressant therapeutic agent. In an even further aspect, the geriatric patient is in about the first 30 days of treatment with an anti-depressant therapeutic agent. In a further aspect, the geriatric patient is older than about 55 years. In a still further aspect, the geriatric patient is older than about 60 years. In a yet further aspect, the geriatric patient is older than about 65 years. In a still further aspect, the geriatric patient is older than about 70 years. In an even further aspect, the geriatric patient is older than about 75 years.

1. Treatment of a Depression Disorder

In one aspect, the invention relates to a method for the treatment of a mammal diagnosed with a depression disorder comprising the step of administering to the mammal an effective amount of at least one creatine analog.

a. Depression Disorders

In one aspect, the depression disorder is selected from major depressive disorder, minor depression disorder, dysthymia, postpartum depression, seasonal affective disorder, bipolar disorder, mixed anxiety depression, unspecified depression, adjustment disorder, atypical depression, psychotic depression, and suicidal ideation. In a further aspect, the depression disorder is major depressive disorder. In an even further aspect, the patient has suicidal ideation. In a further aspect, the patient is under about 25 years of age. In a still further aspect, the patient is an adolescent. In an even further aspect, the patient is about 13 to about 18 years of age. In a further aspect, the patient is an adolescent that is resistant to treatment with a selective serotonin reuptake inhibitor. In a still further aspect, the adolescent is female.

In a further aspect, the depression disorder is diagnosed in a subject who is selected an adolescent, a geriatric patient, an individual with mild to traumatic brain injury, a military veteran with a history of post-traumatic stress disorder, a military veteran with combat experience, a military veteran with mild to severe traumatic brain injury, a individual less than about 25 years of age who is prescribed an anti-depressant therapeutic agent, and an individual who has a history of substance abuse.

b. Dosages

Typically, a creatine analog is administered in an effective amount. In a further aspect, the effective amount is a therapeutically effective amount. In a further aspect, the effective amount is a prophylactically effective amount. In a further aspect, the effective amount is a synergistically effective amount.

In a further aspect, the creatine analog and one or more agents are together administered in a therapeutically effective amount.

In a further aspect, the effective amount is from about 0.5 to about 50 g/day, for example, from about 1 to about 20 g/day, from about 1 to about 10 g/day, from about 1 to about 8 g/day, from about 1 to about 5 g/day, from about 2 to about 10 g/day, from about 2 to about 8 g/day, from about 2 to about 5 g/day, from about 3 to about 10 g/day, from about 3 to about 8 g/day, or from about 3 to about 5 g/day. In a further aspect, the effective amount is about 1 g/day, about 2 g/day, about 3 g/day, about 4 g/day, about 5 g/day, about 6 g/day, about 7 g/day, about 8 g/day, about 9 g/day, or about 10 g/day.

In one aspect, the amount can be given once per day. In a further aspect, half of the amount can be given twice per day. In a further aspect, one-third of the amount can be given three times per day.

2. Agents Having a Side Effect of Causing Depression, Suicide, or Suicidal Ideation

In one aspect, the agent known to have a side effect of causing depression is selected from an anticonvulsant, a barbiturate, a benzodiazepine, a β-adrenergic blocker, a calcium channel blocker, an estrogen, a fluoroquinolone, an interferon alpha, an opiod, and a statin. In a further aspect, the agent known to have a side effect of causing depression, suicide or suicidal ideation is selected from Abilify® (aripiprazole), Accutane® (isotretinoin), Ambien® (zolpidem), Antabuse® (disulfuram), Chantix® (varenicline), Lariam® (mefloquine), Norplant® (levonorgestrel Implant), Parlodel® (bromocriptine), Savella® (milnacipran), Singulair® (montelukast), Strattera® (atomoxetine), tetrabenazine, tramadol, and Zovirax® (acyclovir). In a still further aspect, the agent known to have a side effect of causing depressin, suicide or suicidal ideation is selected from Adderall® (amphetamine and dextroamphetamine), Benzedrine® (amphetamine sulfate), Concerta® (methylphenidate), Cylert® (pemoline), Daytrana® (methylphenidate), Desoxyn® (methamphetamine), Dexedrine® (dextroamphetamine), Dextrostat® (dextroamphetamine), Equasym® (methylphenidate), Focalin® (dexmethylphenidate), Metadate® (methylphenidate), Methylin® (methylphenidate hydrochloride), Provigil® (modafinil), Ritalin® (methylphenidate), and Vyvanse® (lisdexamphetamine). In a further aspect, the agent known to have a side effect of causing depressin, suicide or suicidal ideation is selected from Tegretol® (carbamazepine), Klonopin® (clonazepam), Depakote® (divalproex), Depakene® (valproic acid), Zarontin® (ethosuximide), Peganone® (ethotoin), Felbatol® (felbamate), Neurontin® (gabapentin), Lamictal® (lamotrigine), Vimpat® (lacosamide), Keppra® (levetiracetam), Mesantoin® (mephenyloin), Celontin® (methsuximide), Trileptal® (oxcarbazepine), Dilantin® (phenyloin), Lyrica® (pregabalin), Mysoline® (primidone), Gabitril® (tiagabine), Topamax® (topiramate), Tridione® (trimethadione), and Zonegran® (zonisamide). In a further aspect, the agent known to have a side effect of causing depression, suicide or suicidal ideation is selected from Elavil® (amitriptyline HCl), Prozac® (fluoxetine), Zoloft® (sertraline), Paxil® (paroxetine), Luvox® (fluvoxamine), Celexa® (citalopram), Lexapro® (escitalopram), Wellbutrin® (bupropion), Effexor® (venlafaxine), Serzone® (nefazodone), Remeron® (mirtazapine), and Norpramin® (desipramine). In various aspects, the following combinations are specifically contemplated:

Agent having a side effect
of causing depression

| Creatine Analog | Chemical Name | Trade Name |
|---|---|---|
| creatine monohydrate, | isotretinoin | Accutane ® |
| creatine ethyl ester, and/or | | |
| magnesium creatine chelate | | |
| creatine monohydrate, | disulfiram | Antabuse ® |
| creatine ethyl ester, and/or | | |
| magnesium creatine chelate | | |
| creatine monohydrate, | bromocriptine | Parlodel ® |
| creatine ethyl ester, and/or | | |
| magnesium creatine chelate | | |
| creatine monohydrate, | levonorgestrel | Norplant ® |
| creatine ethyl ester, and/or | | |
| magnesium creatine chelate | | |
| creatine monohydrate, | acyclovir | Zovirax ® |
| creatine ethyl ester, and/or | | |
| magnesium creatine chelate | | |
| creatine monohydrate, | varenicline | Chantix ® |
| creatine ethyl ester, and/or | | |
| magnesium creatine chelate | | |
| creatine monohydrate, | montelukast | Singulair ® |
| creatine ethyl ester, and/or | | |
| magnesium creatine chelate | | |
| creatine monohydrate, | amphetamine and | Adderall ® |
| creatine ethyl ester, and/or | dextroamphetamine | |
| magnesium creatine chelate | | |
| creatine monohydrate, | Methylphenidate | Ritalin ® |
| creatine ethyl ester, and/or | | |
| magnesium creatine chelate | | |
| creatine monohydrate, | Clonazepam | Klonopin ® |
| creatine ethyl ester, and/or | | |
| magnesium creatine chelate | | |
| creatine monohydrate, | Carbamazepine | Tegretol ® |
| creatine ethyl ester, and/or | | |
| magnesium creatine chelate | | |
| creatine monohydrate, | gabapentin | Neurontin ® |
| creatine ethyl ester, and/or | | |
| magnesium creatine chelate | | |
| creatine monohydrate, | topiramate | Topamax ® |
| creatine ethyl ester, and/or | | |
| magnesium creatine chelate | | |

3. Agent Known to Treat a Depression Disorder

In one aspect, the agent known to treat a depression disorder is selected from selective serotonin reuptake inhibitor, serotonin-norepinephrine reuptake inhibitors, tricyclic antidepressants, tetracyclic antidepressants, phenylpiperazine antidepressants, monoamine oxidase inhibitors, and atypical antidepressants. In a still further aspect, the agent known to treat a depression disorder is selected from fluoxetine, norfluoxetine, escitalopram, citalopram, paroxetine, fluvoxamine, sertraline, reboxetine, nisoxetine, zimelidine, litoxetine, indalpine, gepirone, femoxetine, alaproclate, and racemic forms or derivatives thereof, and pharmaceutically acceptable salt thereof. In a further aspect, the agent known to treat a depression disorder is selected from venlafaxine, desvenlafaxine, duloxetine, and milnacipran. In a further aspect, the agent known to treat a depression disorder is selected from amoxapine, imipramine, trimipramine, nortriptyline, clomipramine, amitriptyline, doxepin, protriptyline, and desipramine. In a further aspect, the agent known to treat a depression disorder is selected from tranylcypromine, isocarboxazid, selegiline, and phenelzine. In a still further aspect, the agent known to treat a depression disorder is selected from mirtazapine, maprotiline, bupropion, aripiprazole, ziprasidone, and agomelatine. In a further aspect, the agent known to treat a depression disorder is a selective serotonin reuptake inhibitor.

In a further aspect, the agent know to treat a depression disorder is selected from serotonin-2 antagonist/reuptake inhibitors, alpha-2 antagonists plus serotonin-2 and serotonin-3 antagonists, serotonin/norepinephrine/dopamine reuptake inhibitors, norepinephrine and dopamine reuptake inhibitors and other antidepressants.

Typically, an agent is administered in an effective amount, per its normal dosing instructions. In one aspect, the effective amount is a therapeutically effective amount. In a further aspect, the effective amount is a prophylactically effective amount. In one aspect, the effective amount is a synergistically effective amount; for example, when combined with a creatine analog, in certain aspects, the agent can be employed in therapeutically effective amounts that are lower than the amount in dictated in its normal dosing instructions.

4. Reducing Risk of Suicide

In one aspect, the invention relates to a method for reducing risk of suicide in a patient having a depression disorder comprising the step of administering to the patient an effective amount of at least one creatine analog. In one aspect, the method further comprised the step of identifying the patient as having a need of suicide risk reduction. In a further aspect, the patient has been diagnosed with a need for treatment of the depression disorder prior to the administering step. In a further aspect, a medical practioner, including, but not limited to, a psychiatrist, medical doctor, psychologist, licensed social worker, nurse, physician assistant, professional counselor, or substance abuse counselor, has determined that the patient has Children's Depression Rating Scale-Revised (CDRS-R) raw score>40. In a yet further aspect, the patient with a Children's Depression Rating Scale-Revised (CDRS-R) raw score>40 has been treated with a selective serotonin reuptake inhibitor for 8 weeks. In a still further aspect, the selective serotonin reuptake inhibitor is fluoxetine. In an even further aspect, the patient has suicidal ideation. In a further aspect, the patient is under about 25 years of age. In a further aspect, the patient is an adolescent. In a yet further aspect, the patient is about 13 to about 18 years of age. In a still further aspect, the patient who is an adolescent is resistant to treatment with a selective serotonin reuptake inhibitor. In an even still further aspect, the adolescent is female. In a further aspect, the patient who is an adolescent has suicidal ideation. In a still further aspect, the patient who is an adolescent has suicidal ideation and is resistant to treatment with a selective serotonin reuptake inhibitor.

In one aspect, the methods and compositions of the invention relate to the use of creatine analogues to reduce the risk of suicide when coadministered with a selective serotonin reuptake inhibitor. In a further aspect, the activity of an selective serotonin reuptake inhibitor is enhanced in an individual in need thereof. In a further aspect, the method comprises co-administering to the individual a creatine analog and a selective serotonin reuptake inhibitor, wherein the creatine analog is administered in an effective amount sufficient to normalize depression symptoms in the individual, thereby resulting in greater activity of the selective serotonin reuptake inhibitor in the individual than would occur in the absence of co-administration of the creatine analogue. In a further aspect, the selective serotonin reuptake inhibitor is citalopram, escitalopram, flouxetine, fluvoxamine, paroxetine, sertraline, trazodone, venlafaxine, mirtazepine, clomipramine, or combinations with other psychotropic medications including an anti-psychotic, an anti-convulsant, a tricyclic antidepressant, a monoamine oxidase inhibitor, a selective serotonin reuptake inhibitor, a selective serotonin-norepinephrine reuptake inhibitor, a norepinephrine dopamine reuptake inhibitor, a serotonin-2 antagonist reuptake inhibitor, a benzodiazepine, a wakefulness promoting agent, anti-manic agent, or a combination of one or more of the foregoing. In a further aspect, coadministered comprises administering a creatine analog at the same time as the selective serotonin reuptake inhibitor. In a yet further aspect, coadministered comprises administering a creatine analog a different time than the selective serotonin reuptake inhibitor.

In one aspect, ³¹P-MRS spectra are acquired from the patient to determine PCr levels. In a still further aspect, the ³¹P-MRS spectra show an increase in PCr levels in the patient upon treatment with a creatine analog. In an even further aspect, the increase in PCr levels in the patient is detected after about 1-4 weeks of treatment with a creatine analog. In a further aspect, the increase in PCr levels in the patient is maintained for at least one week upon discontinuation of treatment with a creatine analog. In a still further aspect, the PCr levels continues to increase for at least one week in the patient upon discontinuation of treatment with a creatine analog. In an even further aspect, there is no change significant change in β-NTP levels, pH or PCr/β-NTP ratio in the patient upon treatment with a creatine analog. In a further aspect, there is no significant difference in the patient in the β-NTP levels, pH or PCr/β-NTP ratio compared to a normal subject.

5. Reducing Likelihood of Depression Symptoms

In one aspect, the invention relates to a method of reducing likelihood of depression symptoms in a subject comprising the step of administering to the patient an effective amount of at least one creatine analog within ten days of administration to the subject an agent known to have a side effect of causing depression. In a further aspect, the method further comprises the step of identifying a subject having a likelihood of depression symptoms. In a further aspect, the subject has been diagnosed with a need for treatment of a depression disorder prior to the administering step.

In one aspect, the methods and compositions of the invention relate to the use of creatine analogues to reduce the likelihood of depression symptoms when coadministered with a selective serotonin reuptake inhibitor within ten days of administration to the subject an agent known to have a side effect of causing depression. In a further aspect, the activity of an selective serotonin reuptake inhibitor is enhanced in an individual in need thereof. In a further aspect, the method comprises co-administering to the individual a creatine analog and a selective serotonin reuptake inhibitor, wherein the creatine analog is administered in an effective amount sufficient to normalize depression symptoms in the individual, thereby resulting in greater activity of the selective serotonin reuptake inhibitor in the individual than would occur in the absence of co-administration of the creatine analogue. In a further aspect, the selective serotonin reuptake inhibitor is citalopram, escitalopram, flouxetine, fluvoxamine, paroxetine, sertraline, trazodone, venlafaxine, mirtazepine, clomipramine, or combinations with other psychotropic medications including an anti-psychotic, an anti-convulsant, a tricyclic antidepressant, a monoamine oxidase inhibitor, a selective serotonin reuptake inhibitor, a selective serotonin-norepinephrine reuptake inhibitor, a norepinephrine dopamine reuptake inhibitor, a serotonin-2 antagonist reuptake inhibitor, a benzodiazepine, a wakefulness promoting agent, anti-manic agent, or a combination of one or more of the foregoing. In a further aspect, coadministered comprises administering a creatine analog at the same time as the selective serotonin reuptake inhibitor. In a yet further aspect, coadministered comprises administering a creatine analog a different time than the selective serotonin reuptake inhibitor.

In a further aspect, the creatine analog is administered within one, two, three, four, five, six, or seven days of administration of the agent known to have a side effect of causing depression.

6. Combination Therapeutics

In one aspect, the invention relates to a method for the treatment of a patient diagnosed with a depression disorder comprising the step of administering to the patient, together in a therapeutically effective amount, at least one creatine analog and at least one agent known to treat a depression disorder. In various aspects, the following combination therapeutics are specifically contemplated:

| | Agent known to treat a depression disorder | |
|---|---|---|
| Creatine Analog | Chemical Name | Trade Name |
| creatine monohydrate, | fluoxetine | Prozac ® |
| creatine ethyl ester, and/or | | |
| magnesium creatine chelate | | |
| creatine monohydrate, | escitalopram | Lexapro ® |
| creatine ethyl ester, and/or | | |
| magnesium creatine chelate | | |
| creatine monohydrate, | citalopram | Celexa ®, Cipramil ® |
| creatine ethyl ester, and/or | | |
| magnesium creatine chelate | | |
| creatine monohydrate, | paroxetine | Paxil ®, Pexeva ® |
| creatine ethyl ester, and/or | | |
| magnesium creatine chelate | | |
| creatine monohydrate, | fluvoxamine | Luvox ® |
| creatine ethyl ester, and/or | | |
| magnesium creatine chelate | | |
| creatine monohydrate, | sertraline | Zoloft ® |
| creatine ethyl ester, and/or | | |
| magnesium creatine chelate | | |
| creatine monohydrate, | venlafaxine | Effexor ® |
| creatine ethyl ester, and/or | | |
| magnesium creatine chelate | | |
| creatine monohydrate, | desvenlafaxine | Pristiq ® |
| creatine ethyl ester, and/or | | |
| magnesium creatine chelate | | |
| creatine monohydrate, | duloxetine | Cymbalta ® |
| creatine ethyl ester, and/or | | |
| magnesium creatine chelate | | |
| creatine monohydrate, | milnacipran | Savella ® |
| creatine ethyl ester, and/or | | |
| magnesium creatine chelate | | |
| creatine monohydrate, | amoxapine | Asendin ® |
| creatine ethyl ester, and/or | | |
| magnesium creatine chelate | | |
| creatine monohydrate, | imipramine | Tofranil ® |
| creatine ethyl ester, and/or | | |
| magnesium creatine chelate | | |
| creatine monohydrate, | trimipramine | Surmontil ® |
| creatine ethyl ester, and/or | | |
| magnesium creatine chelate | | |
| creatine monohydrate, | nortriptyline | Aventyl ®, Pamelor ® |
| creatine ethyl ester, and/or | | |
| magnesium creatine chelate | | |
| creatine monohydrate, | clomipramine | Anafranl ®, |
| creatine ethyl ester, and/or | | Clomicalm ® |
| magnesium creatine chelate | | |
| creatine monohydrate, | amitriptyline | Elavil ®, Endeep ®, |
| creatine ethyl ester, and/or | | Vanatrip ® |
| magnesium creatine chelate | | |
| creatine monohydrate, | doxepin | Adapin ®, Prudoxin ®, |
| creatine ethyl ester, and/or | | Silenor ®, Sinequan ®, |
| magnesium creatine chelate | | Zonalon ® |
| creatine monohydrate, | protriptyline | Vivactil ® |
| creatine ethyl ester, and/or | | |
| magnesium creatine chelate | | |
| creatine monohydrate, | desipramine | Norpramin ® |
| creatine ethyl ester, and/or | | |
| magnesium creatine chelate | | |
| creatine monohydrate, | tranylcypromine | Parnate ® |
| creatine ethyl ester, and/or | | |
| magnesium creatine chelate | | |
| creatine monohydrate, | isocarboxazid | Marplan ® |
| creatine ethyl ester, and/or | | |
| magnesium creatine chelate | | |
| creatine monohydrate, | selegiline | Anipryl ®, Atapryl ®, |
| creatine ethyl ester, and/or | | Carbex ®, Eldepryl ®, |
| magnesium creatine chelate | | Emsam ®, Zelapar ® |
| creatine monohydrate, | phenelzine | Nardil ® |
| creatine ethyl ester, and/or | | |
| magnesium creatine chelate | | |

In one aspect, the methods and compositions of the invention relate to the use of creatine analogues to increase the efficacy of a selective serotonin reuptake inhibitor. In a further aspect, the activity of an selective serotonin reuptake inhibitor is enhanced in an individual in need thereof. In a further aspect, the method comprises co-administering to the individual a creatine analog and a selective serotonin reuptake inhibitor, wherein the creatine analog is administered in an effective amount sufficient to normalize depression symptoms in the individual, thereby resulting in greater activity of the selective serotonin reuptake inhibitor in the individual than would occur in the absence of co-administration of the creatine analogue. In a further aspect, the selective serotonin reuptake inhibitor is citalopram, escitalopram, flouxetine, fluvoxamine, paroxetine, sertraline, trazodone, venlafaxine, mirtazepine, clomipramine, or combinations with other psychotropic medications including an anti-psychotic, an anti-convulsant, a tricyclic antidepressant, a monoamine oxidase inhibitor, a selective serotonin reuptake inhibitor, a selective serotonin-norepinephrine reuptake inhibitor, a norepinephrine dopamine reuptake inhibitor, a serotonin-2 antagonist reuptake inhibitor, a benzodiazepine, a wakefulness promoting agent, anti-manic agent, or a combination of one or more of the foregoing. In a further aspect, coadministered comprises administering a creatine analog at the same time as the selective serotonin reuptake inhibitor. In a yet further aspect, coadministered comprises administering a creatine analog a different time than the selective serotonin reuptake inhibitor.

In one aspect, the invention relates to an oral dosage form comprising at least one creatine analog and one or more of at least one agent known to treat a depression disorder; or at least one agent known to have a side effect of causing depression.

7. Treatment of SSRI-Treatment Resistant Patients

In one aspect, the invention relates to a method for the treatment of a depression disorder in a selective serotonin reuptake inhibitor-treatment resistant patient comprising the step of administering to the mammal an effective amount of a selective serotonin reuptake inhibitor and an effective amount of at least one creatine analog. In a further aspect, the method further comprises the step of identifying a patient having a likelihood of depression symptoms. In a further aspect, the patient has been diagnosed with a need for treatment of a depression disorder prior to the administering step. In a further aspect, the method further comprises the step of identifying a patient having resistance to selective serotonin reuptake inhibitor treatment. In a further aspect, the patient has been diagnosed with resistance to selective serotonin reuptake inhibitor treatment prior to the administering step. In a further aspect, a medical practioner, including, but not limited to, a psychiatrist, medical doctor, psychologist, licensed social worker, nurse, physician assistant, professional counselor, or substance abuse counselor, has determined that the patient has Children's Depression Rating Scale-Revised (CDRS-R) raw score>40. In a yet further aspect, the patient with a Children's Depression Rating Scale-Revised (CDRS-R) raw score>40 has been treated with a selective serotonin reuptake inhibitor for 8 weeks. In a still further aspect, the selective serotonin reuptake inhibitor is fluoxetine. In an even further aspect, the patient has suicidal ideation. In a further aspect, the patient is under about 25 years of age. In a still further aspect, the patient who is an adolescent is resistant to treatment with a selective serotonin reuptake inhibitor. In an even still further aspect, the adolescent is female. In a further aspect, the patient who is an adolescent has suicidal ideation. In a still further aspect, the patient who is an adolescent has suicidal ideation and is resistant to treatment with a selective serotonin reuptake inhibitor.

In one aspect, the methods and compositions of the invention relate to the use of creatine analogues to treat patients who are resistant to treatment with a selective serotonin reuptake inhibitor. In a further aspect, the activity of an selective serotonin reuptake inhibitor is enhanced in an individual in need thereof. In a further aspect, the method comprises co-administering to the individual a creatine analog and a selective serotonin reuptake inhibitor, wherein the creatine analog is administered in an effective amount sufficient to normalize depression symptoms in the individual, thereby resulting in greater activity of the selective serotonin reuptake inhibitor in the individual than would occur in the absence of co-administration of the creatine analogue. In a further aspect, the selective serotonin reuptake inhibitor is citalopram, escitalopram, flouxetine, fluvoxamine, paroxetine, sertraline, trazodone, venlafaxine, mirtazepine, clomipramine, or combinations with other psychotropic medications including an anti-psychotic, an anti-convulsant, a tricyclic antidepressant, a monoamine oxidase inhibitor, a selective serotonin reuptake inhibitor, a selective serotonin-norepinephrine reuptake inhibitor, a norepinephrine dopamine reuptake inhibitor, a serotonin-2 antagonist reuptake inhibitor, a benzodiazepine, a wakefulness promoting agent, anti-manic agent, or a combination of one or more of the foregoing. In a further aspect, coadministered comprises administering a creatine analog at the same time as the selective serotonin reuptake inhibitor. In a yet further aspect, coadministered comprises administering a creatine analog a different time than the selective serotonin reuptake inhibitor.

In one aspect, the selective serotonin reuptake inhibitor and the creatine are administered substantially simultaneously. In a further aspect, the creatine analog is administered within ten days of administration of the selective serotonin reuptake inhibitor. In a further aspect, the creatine analog is administered within one, two, three, four, five, six, or seven days of administration of the selective serotonin reuptake inhibitor.

In one aspect, ³¹P-MRS spectra are acquired from the patient to determine PCr levels. In a still further aspect, the ³¹P-MRS spectra show an increase in PCr levels in the patient upon treatment with a creatine analog. In an even further aspect, the increase in PCr levels in the patient is detected after about 1-4 weeks of treatment with a creatine analog. In a further aspect, the increase in PCr levels in the patient is maintained for at least one week upon discontinuation of treatment with a creatine analog. In a still further aspect, the PCr levels continues to increase for at least one week in the patient upon discontinuation of treatment with a creatine analog. In an even further aspect, there is no change significant change in β-NTP levels, pH or PCr/β-NTP ratio in the patient upon treatment with a creatine analog. In a further aspect, there is no significant difference in the patient in the β-NTP levels, pH or PCr/β-NTP ratio compared to a normal subject.

8. Diagnosing and Treating a Depression Disorder

In one aspect, the invention relates to a method for the treatment of a subject comprising the steps of diagnosing the subject as having a depression disorder; and administering to the subject an effective amount of at least one creatine analog.

In one aspect, diagnosing comprises performing a ³¹P MRS experiment upon the subject and identifying a level of a metabolic marker. In a further aspect, a metabolic marker comprises at least one of magnesium, pH, total nucleoside concentration, phosphocreatine, and β nucleoside triphosphate.

In one aspect, a method of diagnosis of a depression disorder comprises determining, in a brain of a patient, levels of a marker (e.g., a metabolite) indicative of a brain bioenergetic metabolic state of the patient, the brain bioenergetic metabolic state being predictive as to whether the patient will manifest reduced symptoms of depression in response to a depression treatment. In a still further aspect, the marker is detected in a region of the brain comprising at least one of the anterior cingulate, the amygdala, and the hippocampus of the brain. In one aspect, the method of diagnosis of depression comprises a mood disorder, such as depression or bipolar disorder. major depressive disorder. In a further aspect, the patient is under 20 years of age.

In one aspect, the marker comprises at least one of adenosine triphosphate, adenosine diphosphate, and phosphocreatine. In a further aspect, the first and second levels of the marker are determined by ³¹P magnetic resonance spectroscopy or MR spectroscopy of another suitable isotope. In a still further aspect, the marker can comprise at least one of magnesium, pH, total nucleoside triphosphate, and β NTP. In a yet further aspect, the marker can also be any other known to those of skill in the art. In an even further aspect, the marker comprises phosphocreatine.

In one aspect, a marker comprises a brain bioenergetic metabolic state marker, wherein the brain energetic metabolic state marker comprises a pH, a compound comprising magnesium, and a compound comprising phosphorus (e.g., PCr, ATP, ADP, Pi, total NTP, α-NTP, β-NTP, γ-NTP, and combinations thereof). In a further aspect, levels of such phosphorus comprising compounds present in the brain of a patient can be determined by, for instance, ³¹P MRS. In a still further aspect, the patient can suffer from major depression disorder. In a further aspect, the patient can suffer from depression resulting from recurring head pain, such as migraine headaches, cluster headaches, and tension headaches. In a still further aspect, the antidepression treatment can comprise administering to the patient an SSRI, a tricyclic, a thyroid hormone, or combinations thereof.

In one aspect, brain levels of ADP, ATP, and PCr are different, as compared to a subject that does not suffer from depression, in the brain of a subject that suffers from depression and that will likely manifest reduced levels and/or symptoms of depression in response to an antidepression treatment. In some embodiments, an antidepression treatment results in a substantial normalization of brain levels of ADP, ATP, and PCr in the brain of a patient that manifests reduced levels and/or symptoms of depression in response to the antidepression treatment. In a further aspect, normalizing changes in brain PCr and ATP levels in can result in the achievement of a substantially normalized brain bioenergetic metabolic state as a result of the buffer role of PCr in relation to ATP. For example, brain concentrations of ATP can, at the expense of brain PCr concentrations, normally be maintained at substantially uniform levels by PCr transfer of a high-energy phosphate group to ADP, re-forming ATP in a reaction mediated by, for example, creatine kinase. A reduction of an brain concentration of ADP, ATP, or PCr to a substantially non-physiologic level can result in a brain metabolic state correlated with depression. An antidepression treatment that substantially normalizes a level of ADP, ATP, or PCr in a patient suffering from depression can thereby alleviate a level or symptom of depression in the patient. But such normalizing changes in brain ADP, ATP, and PCr brain concentrations in patients that respond to a depression treatment can also be achieved by other mechanisms.

In one aspect, a mitochondrial dysfunction characterizes a patient that manifests reduced levels and/or symptoms of depression in response to an antidepression treatment modality. In a further aspect, low levels of magnesium in the brain of a subject that suffers from depression, as compared with normal subjects, can result from impaired oxidative phosphorylation related to mitochondrial dysfunction; and impaired oxidative phosphorylation can result in a brain bioenergetic metabolic state correlated with depression. An antidepression treatment that substantially normalizes, in a patient suffering from depression, brain magnesium levels resulting from mitochondrial dysfunction can alleviate a level or symptom of depression in the patient. But such normalizing changes in levels of magnesium in the brain of a patient that responds to a depression treatment modality can also be achieved by other mechanisms.

In one aspect, diagnosis of a depression disorder comprises a medical history. In a further aspect, symptoms of depression can include, for example, depressive mood, hypobulia, loss of interest and pleasure, disrupted concentration and attention, lowered self-esteem and self-confidence, feelings of guilt and worthlessness, pessimism about the future, thoughts of suicide, sleep disorders, and loss of appetite. These symptoms have features peculiar to depression, which differ from depressed feelings experienced by anyone, and also differ from the lowered mental activity and sense of exhaustion experienced by people afflicted with physical diseases. The symptoms of depression are mainly comprehended by taking a precise medical history, questioning when and how the symptoms in terms of mental activity were developed and what types of damages have been imposed upon their social and domestic lives, and confirming various symptoms based on a patient's attitude or the contents of conversations during consultation. For example, family medical history, anamnesis, physical conditions, early developmental history, life history, personality inclination, premorbid social adaptation, and the occurrence of any episode(s) that had triggered the disease can be important references. In order to accurately comprehend these factors, an interview needs to be conducted by a highly skilled specialist in psychiatric medicine for approximately 1 hour. Further, it should be confirmed that a patient does not have any major abnormalities in terms of general physical or neurological conditions. If necessary, the possibility of the existence of organic brain disorders is to be eliminated by electroencephalography or brain imaging tests. The patient is then subjected to diagnosis.

In one aspect, the diagnosis comprises comparing the findings of the medical history with the diagnostic standards. Diagnostic standards comprise those provided in DSM-IV: Diagnostic and Statistical Manual of Mental Disorders—Fourth Edition, Text Revision (American Psychiatric Association, 2000). In a further aspect, the diagnostic standards provided in the International Statistical Classification of Diseases and Related Health Problems 10th Revision (ICD-10) published by the World Health Organization.

In one aspect, a medical practioner, including, but not limited to, a psychiatrist, medical doctor, psychologist, licensed social worker, nurse, physician assistant, professional counselor, or substance abuse counselor, can use a “depression symptoms rating scale”. The term “depressions symptoms rating scale” means any one of a number of standardized questionnaires, clinical instruments, or symptom inventories utilized to measure symptoms and symptom severity in depression. Such rating scales are often used in clinical practice to assess a subject or assist in providing a diagnosis. Such rating scales are often used in clinical studies to define treatment outcomes, based on changes from the study's entry point(s) to endpoint(s). In further aspect, a depression symptoms rating scale comprises one or more of Apathy Scale of Glenn et al., Bech-Rafaelsen Melancholia Scale, Beck Depression Inventory (BDI), Beck Depression Inventory II (BDI-II), Brief Screening Instrument of Fabacher et al to Detect Depression in an Elderly Patient in the Emergency Department (ED-DSI), Burns Depression Checklist (BDC), Center for Epidemiologic Studies Depression Scale—Revised (CESD-R), Center for Epidemiologic Studies Depression Scale (CES-D), Center for Epidemiological Studies Depression Scale for Children (CES-DC), Children's Depression Inventory (CDI), Children's Depression Rating Scale, Revised (CDRS-R), Clinical Global Impression Scale-I, Clinician Administered Posttraumatic Stress Disorder (PTSD) Scale-2 (CAPS), Cornell Scale for Depression in Dementia (CSDD), Depression and Anxiety in Youth Scale (DAYS), Depression Anxiety Stress Scales (DASS), Depression Outcomes Module (DOM), Diagnostic and Statistical Manual of Mental Disorders 4th Edition (DSM IV), Edinburgh Postnatal Depression Scale (EPDS), Geriatric Depression Scale (GDS; long or short format), Global Assessment of Functioning Scale, Goldberg Depression & Mania Scales, Hamilton Anxiety Rating Scale, Hamilton Depression Rating Scale (HDRS), Hamilton Depression Scale (HAM-D), Harvard National Depression Screening Scale (HANDS), Hospital Anxiety and Depression Scale (HADS), International Statistical Classification of Diseases and Related Health Problems 10th Revision (ICD-10), K-SADS Depression Rating Scale (K-DEP), KSADS Lifetime and Present (KSADS-PL) schedule, Liebowitz Social Anxiety Scale, Major Depression Inventory (MDI), Medical Outcomes Study Depression Questionnaire, Mehrabian Trait Anxiety and Depression Scales, Mini-Mental State Examination, Montgomery-Asberg Depression Rating Scale, Multiscore Depression Inventory (MDI), Multiscore Depression Inventory for Children (MDI-C), Newcastle Diagnostic and ECT Scales of Carney et al for Depression, Online Depression Screening Test (ODST), Panic Disorder Severity Scale, Postpartum Depression Screening Scale (PDSS), Post-Stroke Depression Rating Scale of Gainotti et al, RAND Self Administered Depression Screener, Raskin Scale or Three-Area Severity of Depression Scale, Revised Hamilton Rating Scale for Depression (RHRSD), Reynolds Adolescent Depression Scale (RADS), Reynolds Adolescent Depression Scale, Second Edition (RADS-2), Reynolds Child Depression Scale (RCDS), Risk Factors of Beck for Postpartum Depression (PPD), Risk Factors of Kivela et al for Predicting Chronic Depression in the Elderly, Sheehan Disability Scale, Treatment Outcome PTSD Scale, Yale-Brown Obsessive Compulsive Scale, and Zung Self-Rated Depression Scale

In one aspect, a medical practioner, including, but not limited to, a psychiatrist, medical doctor, psychologist, licensed social worker, nurse, physician assistant, professional counselor, or substance abuse counselor, can use a “depression symptoms rating scale”, can use a “suicide symptoms rating scale”. The term “depressions symptoms rating scale” means any one of a number of standardized questionnaires, clinical instruments, or symptom inventories utilized to measure symptoms and symptom severity in depression. Such rating scales are often used in clinical practice to assess a subject or assist in providing a diagnosis. Such rating scales are often used in clinical studies to define treatment outcomes, based on changes from the study's entry point(s) to endpoint(s). In further aspect, a suicide symptoms rating scale comprises one or more of Adolescent Inventory of Suicide Orientation-30 (ISO-30), Adult Suicidal Ideation Questionnaire (ASIQ), Beck Hopelessness Scale (BHS), Beck Scale for Suicide Ideation (BSS), Child Suicide Risk Assessment (CSRA), Child-Adolescent Suicidal Potential Index (CASPI), Columbia Classification Algorithm of Suicide Assessment (C-CASA), Columbia Suicide Severity Rating Scale (C-SSRS), Coping Inventory for Stressful Situations (CISS), Firestone Assessment of Self-Destructive Thoughts (FAST), Lazurus' BASIC ID tool, Lifetime Parasuicide Count (LPC), MAST—Attraction to Death (MAST-AD), MAST—Repulsion by Life (MAST-RL), Modified SAD PERSONS Scale of Hockberger and Rothstein, Multi-Attitude Suicide Tendency Scale (MAST), Parasuicide History Interview (PHI), Positive and Negative Suicide Ideation Inventory (PANSI), Reasons for Living Inventory (RFL; either long form or short form), Reasons for Living Inventory for Adolescents (RFL-A), Reasons for Living Inventory for Young Adults (RFL-YA), Risk Factors of Powell et al for Predicting the Risk of Suicide in a Psychiatric Ward Inpatient, Risk-Rescue Rating (of Weisman and Worden for Suicide Assessment), Scale for Suicidal Ideation (SSI), Suicidal Behavior History Form (SBHF), Suicidal Behavior Questionnaire for Children (SBQ-C), Suicidal Ideation Questionnaire (SIQ), Suicidal Tendencies Test, Suicide Behaviors Questionnaire (SBQ), Suicide Behaviors Questionnaire-Revised (SBQ-R), Suicide Probability Scale (SPS), and Suicide Resilience Inventory-25 (SRI-25). In a still further aspect, a suicide symptom rating scale comprises a revision, new edition, or derivation of one of a suicide symptom rating scale.

9. Use of Compositions

Also provided are the uses of the disclosed compositions and products. In one aspect, the use relates to a treatment of a disorder in a mammal. In one aspect, the use is characterized in that the mammal is a human. In one aspect, the use is characterized in that the disorder is a neurological and/or psychiatric disorder associated with glutamate dysfunction. In one aspect, the use relates to negative allosteric modulation of metabotropic glutamate receptor activity in a mammal

10. Kits

In one aspect, the invention relates to a kit comprising at least one creatine analog and one or more of at least one agent known to treat a depression disorder; at least one agent known to have a side effect of causing depression; or instructions for treating a disorder associated with depression. In a further aspect, the at least one compound or the at least one product and the at least one agent are co-formulated. In a further aspect, the at least one compound or the at least one product and the at least one agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

11. Manufacture of a Medicament

In one aspect, the invention relates to a method for the manufacture of a medicament for treatment of depression disorder in a mammal comprising combining a therapeutically effective amount of a analog, alone or in combination with another agent, with a pharmaceutically acceptable carrier or diluent.

12. Non-Medical Uses

Also provided are the uses of the disclosed compounds and products as pharmacological tools in the development and standardization of in vitro and in vivo test systems for the evaluation of the effects of therapeutic agents to treat depression disorders in laboratory animals such as cats, dogs, rabbits, monkeys, rats and mice, as part of the search for new therapeutic agents of depression disorders. In a further aspect, the invention relates to the use of a disclosed compound or a disclosed product as pharmacological tools in the development and standardization of in vitro and in vivo test systems for the evaluation of the effects of potentiators of depression disorders in laboratory animals such as cats, dogs, rabbits, monkeys, rats and mice, as part of the search for new therapeutic agents of depression disorders.

### F. REFERENCES

Allen P J, D'Anci K E, Kanarek R B, Renshaw P F (2010) Chronic creatine supplementation alters depression-like behavior in rodents in a sex-dependent manner. Neuropsychopharmacology 35 (2):534-546.
American Psychiatric Association (2000) Diagnostic and statistical manual of mental disorders, fourth edition, text revision. 4th—Text Revision edn. American Psychiatric Association, Washington, D.C.
Barbui C, Esposito E, Cipriani A (2009) Selective serotonin reuptake inhibitors and risk of suicide: A systematic review of observational studies. CMAJ 180 (3):291-297.
Barratt A, Wyer P C, Hatala R, McGinn T, Dans A L, Keitz S, Moyer V, For G G (2004) Tips for learners of evidence-based medicine: 1. Relative risk reduction, absolute risk reduction and number needed to treat. CMAJ 171 (4):353-358.
Bergemann E R, Boles R G (2010) Maternal inheritance in recurrent early-onset depression. Psychiatr Genet 20 (1):31-34.
Birmaher B, Brent D, Bernet W, Bukstein O, Walter H, Benson R S, Chrisman A, Farchione T, Greenhill L, Hamilton J, Keable H, Kinlan J, Schoettle U, Stock S, Ptakowski K K, Medicus J (2007) Practice parameter for the assessment and treatment of children and adolescents with depressive disorders. J Am Acad Child Adolesc Psychiatry 46 (11):1503-1526.
Birmaher B, Ryan N D, Williamson D E, Brent D A, Kaufman J, Dahl R E, Perel J, Nelson B (1996) Childhood and adolescent depression: A review of the past 10 years. Part i. J Am Acad Child Adolesc Psychiatry 35 (11):1427-1439.
Brammer M (2009) The role of neuroimaging in diagnosis and personalized medicine—current position and likely future directions. Dialogues Clin Neurosci 11 (4):389-396.
Brent D, Emslie G, Clarke G, Wagner K D, Asarnow J R, Keller M, Vitiello B, Ritz L, Iyengar S, Abebe K, Birmaher B, Ryan N, Kennard B, Hughes C, DeBar L,
McCracken J, Strober M, Suddath R, Spirito A, Leonard H, Melhem N, Porta G, Onorato M, Zelazny J (2008) Switching to another ssri or to venlafaxine with or without cognitive behavioral therapy for adolescents with ssri-resistant depression: The tordia randomized controlled trial. JAMA 299 (8):901-913.
Bridge J A, Birmaher B, Iyengar S, Barbe R P, Brent D A (2008) Placebo response in randomized controlled trials of antidepressants for pediatric major depressive disorder. Am J Psychiatry:appi.ajp.2008.08020247.
Brosnan J T, Brosnan M E (2007) Creatine: Endogenous metabolite, dietary, and therapeutic supplement. Annu Rev Nutr 27:241-261.
de Leon J (2009) Pharmacogenomics: The promise of personalized medicine for cns disorders. Neuropsychopharmacology 34 (1):159-172.
Drevets W C, Price J L, Furey M L (2008) Brain structural and functional abnormalities in mood disorders: Implications for neurocircuitry models of depression. Brain Struct Funct 213 (1-2): 93-118.
Dubicka B, Hadley S, Roberts C (2006) Suicidal behaviour in youths with depression treated with new-generation antidepressants: Meta-analysis. Br J Psychiatry 189:393-398.
Eaton D K, Kann L, Kinchen S, Shanklin S, Ross J, Hawkins J, Harris W A, Lowry R, McManus T, Chyen D, Lim C, Whittle L, Brener N D, Wechsler H (2010) Youth risk behavior surveillance—united states, 2009. MMWR Surveill Summ 59 (5):1-142.
Fattal O, Link J, Quinn K, Cohen B H, Franco K (2007) Psychiatric comorbidity in 36 adults with mitochondrial cytopathies. CNS Spectr 12 (6):429-438.
Fergusson D M, Horwood U, Ridder E M, Beautrais A L (2005) Subthreshold depression in adolescence and mental health outcomes in adulthood. Arch Gen Psychiatry 62 (1):66-72.
Fergusson D M, Woodward U (2002) Mental health, educational, and social role outcomes of adolescents with depression. Arch Gen Psychiatry 59 (3):225-231.
Fombonne E, Wostear G, Cooper V, Harrington R, Rutter M (2001a) The maudsley long-term follow-up of child and adolescent depression. 1. Psychiatric outcomes in adulthood. Br J Psychiatry 179:210-217.
Fombonne E, Wostear G, Cooper V, Harrington R, Rutter M (2001b) The maudsley long-term follow-up of child and adolescent depression. 2. Suicidality, criminality and social dysfunction in adulthood. Br J Psychiatry 179:218-223.
Food and Drug Administration (1996) Guidance for industry. E6 good clinical practice: Consolidated guidance. Rockville, Md.
Garrison C Z, Waller J L, Cuffe S P, McKeown R E, Addy C L, Jackson K L (1997) Incidence of major depressive disorder and dysthymia in young adolescents. J Am Acad Child Adolesc Psychiatry 36 (4):458-465.
Gerber A J, Peterson B S (2008) Applied brain imaging. J Am Acad Child Adolesc Psychiatry 47 (3):239.
Gerretsen P, Muller D J, Tiwari A, Mamo D, Pollock B G (2009) The intersection of pharmacology, imaging, and genetics in the development of personalized medicine. Dialogues Clin Neurosci 11 (4):363-376.
Goodman W K (2009) Research on biomarkers for mental disorders. National Institute of Mental Health. http://www.nimh.nih.gov/research-funding/grants/concept-clearances/2009/research-on-biomarkers-for-mental-disorders.shtml?WT.mcid=rss. Accessed Aug. 1, 2009
Goodman W K, Murphy T K, Storch E A (2007) Risk of adverse behavioral effects with pediatric use of antidepressants. Psychopharmacology (Berl) 191 (1):87-96.
Gorman J M (2006) Gender differences in depression and response to psychotropic medication. Gend Med 3 (2):93-109.
Gualano B, Artioli G G, Poortmans J R, Lancha Junior A H (2010) Exploring the therapeutic role of creatine supplementation. Amino Acids 38 (1):31-44.
Guy W (1976) ECDEU assessment manual for psychopharmacology, revised. U.S. Dept. of Health, Education, and Welfare, Public Health Service, Alcohol, Drug Abuse, and Mental Health Administration, National Institute of Mental Health, Psychopharmacology Research Branch, Division of Extramural Research Programs, Rockville, Md.
Hammad T A, Laughren T, Racoosin J (2006) Suicidality in pediatric patients treated with antidepressant drugs. Arch Gen Psychiatry 63 (3):332-339.
Hankin B L, Abramson L Y, Moffitt T E, Silva P A, McGee R, Angell K E (1998) Development of depression from preadolescence to young adulthood: Emerging gender differences in a 10-year longitudinal study. J Abnorm Psychol 107 (1):128-140.
Harris G (2008) F.D.A. Requiring suicide studies in drug trials. New York Times, Jan. 24, 2008, p A1
Hetrick S, Merry S, McKenzie J, Sindahl P, Proctor M (2007) Selective serotonin reuptake inhibitors (ssris) for depressive disorders in children and adolescents. Cochrane Database Syst Rev (3):31.
Holsboer F (2008) How can we realize the promise of personalized antidepressant medicines? Nat Rev Neurosci 9 (8):638-646.
Iosifescu D V, Bolo N R, Nierenberg A A, Jensen J E, Fava M, Renshaw P F (2008) Brain bioenergetics and response to triiodothyronine augmentation in major depressive disorder. Biol Psychiatry 63 (12): 1127-1134.
Iosifescu D V, Renshaw P E (2003) 31 p-magnetic resonance spectroscopy and thyroid hormones in major depressive disorder: Toward a bioenergetic mechanism in depression? Harv Rev Psychiatry 11 (2):51-63.
Jost C R, Van Der Zee C E, In 't Zandt H J, Oerlemans F, Verheij M, Streijger F, Fransen J, Heerschap A, Cools A R, Wiering a B (2002) Creatine kinase b-driven energy transfer in the brain is important for habituation and spatial learning behaviour, mossy fibre field size and determination of seizure susceptibility. Eur J Neurosci 15 (10):1692-1706.
Kaptsan A, Odessky A, Osher Y, Levine J (2007) Lack of efficacy of 5 grams daily of creatine in schizophrenia: A randomized, double-blind, placebo-controlled trial. J Clin Psychiatry 68 (6):881-884.
Kasahara T, Kubota M, Miyauchi T, Noda Y, Mouri A, Nabeshima T, Kato T (2006) Mice with neuron-specific accumulation of mitochondrial DNA mutations show mood disorder-like phenotypes. Mol Psychiatry 11 (6):577-593, 523.
Kato T (2007) Mitochondrial dysfunction as the molecular basis of bipolar disorder: Therapeutic implications. CNS Drugs 21 (1):1-11.
Kaufman J, Birmaher B, Brent D, Rao U, Flynn C, Moreci P, Williamson D, Ryan N (1997) Schedule for affective disorders and schizophrenia for school-age children-present and lifetime version (k-sads-pl): Initial reliability and validity data. J Am Acad Child Adolesc Psychiatry 36 (7):980-988.
Kessler R C, McGonagle K A, Swartz M, Blazer D G, Nelson C B (1993) Sex and depression in the national comorbidity survey. I: Lifetime prevalence, chronicity and recurrence. J Affect Disord 29 (2-3):85-96.
Kessler R C, Walters E E (1998) Epidemiology of dsm-iii-r major depression and minor depression among adolescents and young adults in the national comorbidity survey. Depress Anxiety 7 (1):3-14.
Klempan T A, Sequeira A, Canetti L, Lalovic A, Ernst C, ffrench-Mullen J, Turecki G (2009) Altered expression of genes involved in atp biosynthesis and gabaergic neurotransmission in the ventral prefrontal cortex of suicides with and without major depression. Mol Psychiatry 14 (2):175-189.
Koene S, Kozicz T L, Rodenburg R J, Verhaak C M, de Vries M C, Wortmann S, van de Heuvel L, Smeitink J A, Morava E (2009) Major depression in adolescent children consecutively diagnosed with mitochondrial disorder. J Affect Disord 114 (1-3):327-332.
Koshy K M, Griswold E, Schneeberger E E (1999) Interstitial nephritis in a patient taking creatine. N Engl J Med 340 (10):814-815.
Kovacs M (1996) Presentation and course of major depressive disorder during childhood and later years of the life span. J Am Acad Child Adolesc Psychiatry 35 (6):705-715.
Kovacs M (1997) The emanuel miller memorial lecture 1994. Depressive disorders in childhood: An impressionistic landscape. J Child Psychol Psychiatry 38 (3):287-298.
Leibenluft E (2008) Skating to where the puck will be: The importance of neuroimaging literacy in child psychiatry. J Am Acad Child Adolesc Psychiatry 47 (11):1213-1216.
Lewinsohn P M, Hops H, Roberts R E, Seeley J R, Andrews J A (1993) Adolescent psychopathology: I. Prevalence and incidence of depression and other dsm-iii-r disorders in high school students. J Abnorm Psychol 102 (1):133-144.
Lewinsohn P M, Rohde P, Seeley J R (1998) Major depressive disorder in older adolescents: Prevalence, risk factors, and clinical implications. Clin Psychol Rev 18 (7):765-794.
Lynch F L, Clarke G N (2006) Estimating the economic burden of depression in children and adolescents. Am J Prev Med 31(6 Suppl 1):S143-151.
Lyoo I K, Kong S W, Sung S M, Hirashima F, Parow A, Hennen J, Cohen B M, Renshaw P F (2003) Multinuclear magnetic resonance spectroscopy of high-energy phosphate metabolites in human brain following oral supplementation of creatine monohydrate. Psychiatry Res 123 (2):87-100.
Ma J, Lee K V, Stafford R S (2005) Depression treatment during outpatient visits by u.S. Children and adolescents. J Adolesc Health 37 (6):434-442.
McCauley E, Myers K, Mitchell J, Calderon R, Schloredt K, Treder R (1993) Depression in young people: Initial presentation and clinical course. J Am Acad Child Adolesc Psychiatry 32 (4):714-722.
McLeish M J, Kenyon G L (2005) Relating structure to mechanism in creatine kinase. Crit Rev Biochem Mol Biol 40 (1):1-20.
Moller H J, Baldwin D S, Goodwin G, Kasper S, Okasha A, Stein D J, Tandon R, Versiani M (2008) Do ssris or antidepressants in general increase suicidality? Wpa section on pharmacopsychiatry: Consensus statement. Eur Arch Psychiatry Clin Neurosci 258 Suppl 3:3-23.
Moore C M, Christensen J D, Lafer B, Fava M, Renshaw P F (1997) Lower levels of nucleoside triphosphate in the basal ganglia of depressed subjects: A phosphorous-31 magnetic resonance spectroscopy study. Am J Psychiatry 154 (1):116-118.
Moretti A, Gorini A, Villa R F (2003) Affective disorders, antidepressant drugs and brain metabolism. Mol Psychiatry 8 (9):773-785.
Murray C J L, Lopez A D (1996) The global burden of disease. Harvard University Press, Cambridge, Mass.
Newman T B (2004) A black-box warning for antidepressants in children? N Engl Med 351 (16):1595-1598.
Olfson M, Marcus S C, Shaffer D (2006) Antidepressant drug therapy and suicide in severely depressed children and adults: A case-control study. Arch Gen Psychiatry 63 (8):865-872.
Owens P L, Thompson J, Elixhauser A, Ryan K (2003) Care of children and adolescents in u.S. Hospitals. Hcup fact books, vol AHRQ Publication No. 04-0004. Agency for Healthcare Research and Quality, Rockville, Md.
Pavuluri M N, Sweeney J A (2008) Integrating functional brain neuroimaging and developmental cognitive neuroscience in child psychiatry research. J Am Acad Child Adolesc Psychiatry 47 (11):1273-1288.
Pine D S, Cohen E, Cohen P, Brook J (1999) Adolescent depressive symptoms as predictors of adult depression: Moodiness or mood disorder? Am J Psychiatry 156 (1):133-135.
Porsolt R D, Le Pichon M, Jalfre M (1977) Depression: A new animal model sensitive to antidepressant treatments. Nature 266 (5604): 730-732.
Posner K (2010) Columbia-suicide severity rating scale (c-ssrs). Center for Suicide Risk Assessment, Columbia University Medical Center. http://cssrs.columbia.edu/index.html. Accessed May 15, 2010 2010
Posner K, Oquendo M, Stanley B, Gould M, Davies M (2004) Suicidality classification project. Food and Drug Administration Psychopharmacologic Drugs Advisory Committee and the Pediatric Advisory Committee.http://www.fda.gov/ohrms/dockets/ac/04/slides/2004-4065S1_{—}06_FDA [symbol] Posner_files/frame.htm. Accessed Dec. 15, 2006
Posner K, Oquendo M A, Gould M, Stanley B, Davies M (2007) Columbia classification algorithm of suicide assessment (c-casa): Classification of suicidal events in the fda's pediatric suicidal risk analysis of antidepressants. Am J Psychiatry 164(7): 1035-1043.
Poznanski E O, Mokros H B (1996) Children's depression rating scale, revised (cdrs-r) manual. Western Psychological Services, Los Angeles, Calif.
Pritchard N R, Kalra P A (1998) Renal dysfunction accompanying oral creatine supplements. Lancet 351 (9111):1252-1253.
Puri B K (2006) Proton and 31-phosphorus neurospectroscopy in the study of membrane phospholipids and fatty acid intervention in schizophrenia, depression, chronic fatigue syndrome (myalgic encephalomyelitis) and dyslexia. Int Rev Psychiatry 18 (2):145-147.
Rae C, Digney A L, McEwan S R, Bates T C (2003) Oral creatine monohydrate supplementation improves brain performance: A double-blind, placebo-controlled, cross-over trial. Proc Biol Sci 270 (1529):2147-2150.
Rango M, Castelli A, Scarlato G (1997) Energetics of 3.5 s neural activation in humans: A ³¹P mr spectroscopy study. Magn Reson Med 38 (6):878-883.
Renshaw P F, Lyoo I K (2008) Creatine augmentation treatment in major depressive disorder subjects. U.S. National Library of Medicine. Available via U.S. National Institutes of Health. http://clinicaltrials.gov/ct2/show/NCT00729755. Accessed Jul. 20, 2010
Renshaw P F, Parow A M, Hirashima F, Ke Y, Moore C M, Frederick Bde B, Fava M, Hennen J, Cohen B M (2001) Multinuclear magnetic resonance spectroscopy studies of brain purines in major depression. Am J Psychiatry 158 (12):2048-2055.
Reynolds C F, 3rd, Lewis D A, Detre T, Schatzberg A F, Kupfer D J (2009) The future of psychiatry as clinical neuroscience. Acad Med 84 (4):446-450.
Rezin G T, Amboni G, Zugno A I, Quevedo J, Streck E L (2008) Mitochondrial dysfunction and psychiatric disorders. Neurochem Res.
Roitman S, Green T, Osher Y, Karni N, Levine J (2007) Creatine monohydrate in resistant depression: A preliminary study. Bipolar Disord 9 (7):754-758.
SAMHSA (2008) Major depressive episode among youths aged 12 to 17 in the united states: 2004 to 2006. The NSDUH Report. Substance Abuse and Mental Health Services Administration, Washington, D.C.
Sappey-Marinier D, Calabrese G, Fein G, Hugg J W, Biggins C, Weiner M W (1992) Effect of photic stimulation on human visual cortex lactate and phosphates using 1 h and 31 p magnetic resonance spectroscopy. J Cereb Blood Flow Metab 12 (4):584-592.
Schlattner U, Tokarska-Schlattner M, Wallimann T (2006) Mitochondrial creatine kinase in human health and disease. Biochimica et Biophysica Acta (BBA)—Molecular Basis of Disease 1762 (2):164-180.
Schneeweiss S, Patrick A R, Solomon D H, Dormuth C R, Miller M, Mehta J, Lee J C, Wang P S (2010) Comparative safety of antidepressant agents for children and adolescents regarding suicidal acts. Pediatrics 125 (5):876-888.
Serene J A, Ashtari M, Szeszko P R, Kumra S (2007) Neuroimaging studies of children with serious emotional disturbances: A selective review. Can J Psychiatry 52 (3):135-145.
Shao L, Martin M V, Watson S J, Schatzberg A, Akil H, Myers R M, Jones E G, Bunney W E, Vawter M P (2008) Mitochondrial involvement in psychiatric disorders. Ann Med 40 (4):281-295.
Shearer M C, Bermingham S L (2008) The ethics of paediatric anti-depressant use: Erring on the side of caution. J Med Ethics 34 (10):710-714.
Stork C, Renshaw P F (2005) Mitochondrial dysfunction in bipolar disorder: Evidence from magnetic resonance spectroscopy research. Mol Psychiatry 10 (10):900-919.
Tsapakis E M, Soldani F, Tondo L, Baldessarini R J (2008) Efficacy of antidepressants in juvenile depression: Meta-analysis. The British Journal of Psychiatry 193 (1):10-17.
Usala T, Clavenna A, Zuddas A, Bonati M (2008) Randomised controlled trials of selective serotonin reuptake inhibitors in treating depression in children and adolescents: A systematic review and meta-analysis. European Neuropsychopharmacology 18 (1):62-73.
Vitiello B, Rohde P, Silva S, Wells K, Casat C, Waslick B, Simons A, Reinecke M, Weller E, Kratochvil C, Walkup J, Pathak S, Robins M, March J (2006) Functioning and quality of life in the treatment for adolescents with depression study (tads). J Am Acad Child Adolesc Psychiatry 45 (12):1419-1426.
Volz H P, Rzanny R, Riehemann S, May S, Hegewald H, Preussler B, Hubner G, Kaiser W A, Sauer H (1998) 31 p magnetic resonance spectroscopy in the frontal lobe of major depressed patients. Eur Arch Psychiatry Clin Neurosci 248 (6):289-295.
Wade T J, Cairney J, Pevalin D J (2002) Emergence of gender differences in depression during adolescence: National panel results from three countries. J Am Acad Child Adolesc Psychiatry 41 (2):190-198.
Wallimann T, Wyss M, Brdiczka D, Nicolay K, Eppenberger H M (1992) Intracellular compartmentation, structure and function of creatine kinase isoenzymes in tissues with high and fluctuating energy demands: The ‘phosphocreatine circuit’ for cellular energy homeostasis. Biochem J 281 (Pt 1):21-40.
Watanabe A, Kato N, Kato T (2002) Effects of creatine on mental fatigue and cerebral hemoglobin oxygenation. Neurosci Res 42 (4):279-285.
Weissman M M, Wolk S, Goldstein R B, Moreau D, Adams P, Greenwald S, Klier C M, Ryan N D, Dahl R E, Wickramaratne P (1999) Depressed adolescents grown up. JAMA 281 (18):1707-1713.
Whittington C J, Kendall T, Fonagy P, Cottrell D, Cotgrove A, Boddington E (2004) Selective serotonin reuptake inhibitors in childhood depression: Systematic review of published versus unpublished data. Lancet 363 (9418):1341-1345.
Wichstrom L (1999) The emergence of gender difference in depressed mood during adolescence: The role of intensified gender socialization. Dev Psychol 35 (1):232-245.
World Health Organization (2008) The global burden of disease: 2004 update., vol 1. 1 edn. WHO Press, Geneva, Switzerland.
Wyss M, Felber S, Skladal D, Koller A, Kremser C, Sperl W (1998) The therapeutic potential of oral creatine supplementation in muscle disease. Med Hypotheses 51 (4):333-336.
Zahn-Waxler C, Shirtcliff E A, Marceau K (2008) Disorders of childhood and adolescence: Gender and psychopathology. Annu Rev Clin Psychol 4:275-303.

### G. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Study Methods

Approval for the study was obtained from the University of Utah Institutional Review Board (IRB). Informed consent consisted of written parental permission as well as written assent from participants. The study was conducted under U.S. Food and Drug Administration (FDA) Investigational New Drug Application #104,586. An external Data Safety and Monitoring Board vested with authority to halt the trial was established, and the study was conducted in accordance of the principles of Good Clinical Practice (GCP).

Participants were recruited through clinician referrals and IRB-approved advertising. Consecutive patients who met inclusion criteria were enrolled. Inclusion criteria included the following: females between 13-18 years of age with a primary diagnosis of MDD and depressive symptoms persisting for >2 weeks at baseline; fluoxetine treatment for >8 weeks with >4 weeks at a dose of >40 mg/day (if doses higher than 20 mg/day were not tolerated, participants could meet inclusion criteria by taking fluoxetine 20 mg/day for >8 weeks); and a current Children's Depression Rating Scale-Revised (CDRS-R) (Poznanski and Mokros 1996) raw score>40. Consistent with the preliminary nature of the study, few exclusion criteria were applied. For example, no restrictions were placed on concomitant medications or psychotherapy. The study's exclusion criteria were: pre-existing renal disease, proteinuria or microalbuminuria at baseline; contraindication to magnetic resonance scanning (e.g. ferromagnetic implants or claustrophobia); primary diagnosis other than MDD; active psychotic symptoms; high risk for suicidal behavior; positive pregnancy test; active alcohol or drug addiction; known or suspected mental retardation; and unstable medical condition.

Diagnoses were established with the Schedule for Affective Disorders and Schizophrenia for School-Age Children-Present and Lifetime Version (K-SADS-PL) (Kaufman et al. 1997). A complete blood count, metabolic panel, lipid profile, thyroid stimulating hormone, urinalysis and urine microalbumin were obtained to establish that participants were in generally good health, and to rule out the presence of undiagnosed medical conditions. The laboratory studies were repeated at the conclusion of treatment, to prospectively identify any abnormalities associated with creatine administration.

Healthy control female adolescents were recruited for comparison ³¹P-MRS scans through IRB-approved advertising. Following informed consent, the K-SADS-PL and CDRS-R were administered to establish the absence of psychiatric disorder(s) or depressive symptoms. Immediately prior to entering the scanner, all participants had a pregnancy test and a urine drug screen.

Participants with depression were treated with fixed-dose Creapure® brand of creatine monohydrate (AlzChem LLC, Trostberg, Germany) 4 grams by mouth daily for 8 weeks. At each study visit, the following rating scales were administered: the CDRS [symbol] R, the Clinical Global Impressions scale (Guy 1976) and the Columbia-Suicide Severity Rating Scale (C-SSRS) (Posner 2010). Adverse events were recorded at each study visit. Change in CDRS-R raw score was the primary outcome measure, with C-SSRS results and treatment-emergent adverse events serving as secondary clinical outcomes. ³¹P-MRS brain scans were acquired with a Siemens 3 Tesla MRS scanner (Siemens AG, Munich, Germany) that is FDA-approved for clinical use. Two dimensional chemical shift imaging free induction decay (2D CSI FID) pulse sequence with an Fourier voxel resolution of 25×25×25 mm3, Field of View (FOV)=200×200×25 mm3, TR/TE=3000/2.3 ms, vector size=1024, bandwidth=2500 Hz, data collection time=11.2 minutes and the number of averages=24 was implemented to collect 2D CSI FID data using a ³¹P/1H double-tuned volume head coil (Clinical MR Solutions LLC, Brookfield, Wis., USA). The proton channel was used for localization, high-resolution anatomic image acquisition and shimming Shimming was performed over the excited volume. 3D high-resolution images used for localization of CSI data were collected using inversion recovery magnetization prepared rapid gradient echo (MP-RAGE) pulse sequence with isotropic 1 mm3 resolution. The imaging parameters were as follows: TR/TE=2000/3.37 ms, FOV=256×192×144 mm3, and matrix size=256×1 92×144, for a total acquisition time of 4.8 minutes. Participants were instructed during the informed consent process, and again before entering the scanner, that they could discontinue scanning at any point if they experienced discomfort (e.g. claustrophobic anxiety). Non-study personnel trained in research patient advocacy attended each scanning session. The study protocol included two ³¹P-MRS scans: one at baseline and a follow-up scan 8 weeks later.

³¹P MRS data were analyzed using jMRUI software (jMRUI version 4.0, European Community). A Hamming filter was applied to reduce signal contamination from neighbouring voxels before performing 2D Fast Fourier Transform (FFT) on the raw data. Nine voxel signals from a slice with a thickness of 25 mm located at the corpus callosum, anterior commissure and posterior commissure were summed after 2D FFT. Each voxel FID was apodized with a 10 Hz exponential line broadening before zero filling and FFT. The zero-order and first-order phase correction was performed in all spectrums.

We calculated the signal amplitudes for individual metabolites using AMARES (Advanced Method for Accurate, Robust and Efficient Spectral fitting of MRS data), which is a non-linear, time domain fitting algorithm within the jMRUI software application. The estimated amplitudes of phosphorus metabolites are reported with respect to the total ³¹P or beta-NTP amplitude within the time domain signals. The ³¹P MRS signal amplitude for each metabolite is directly related to the integrated area under the modeled spectrum in the frequency domain.

2. Clinical Results

Summary results for each study participant are presented in Table 1 below. All participants were Caucasian; one was Hispanic. None of the participants initiated or terminated psychosocial treatment during the study. Five participants completed the full eight weeks of treatment with creatine and two ³¹P-MRS scans. No participant withdrew from the study due to treatment emergent adverse effects or lack of efficacy. Participants' dose of fluoxetine remained constant during treatment with creatine.

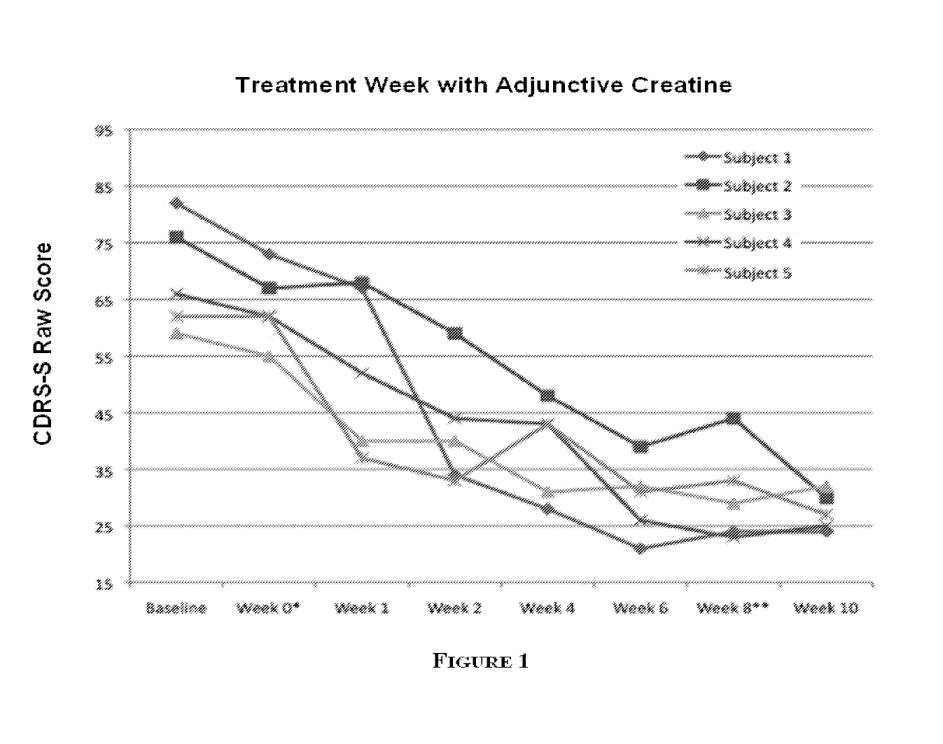

The mean CDRS-R raw score at entry was 69. After eight weeks of treatment with adjunctive creatine the mean CDRS-R score was 30.6. The mean reduction in the primary outcome measure was 38.4, a decrease of 56%. The individual participants' CDRS-R scores during the eight-week treatment period and two-week follow-up period are depicted graphically in FIG. 1. Notably, two weeks after discontinuation of creatine, treatment gains were sustained and the mean CDRS-R raw score at week ten was lower than at the conclusion of treatment.

TABLE 1
Summary of Open-Label Adjunctive Creatine Results.

| | Age | | CDRS-R | CDRS-R | Adverse | C-SSRS | C-SSRS During Treatment | Concomitant |
|---|---|---|---|---|---|---|---|---|
| # | Sex | Diagnoses | Baseline | (Week) | Events | Baseline | With Creatine (Weeks) | Medications |
| 1 | 16 F | MDD | 82 | 24 (8) | Tremor* | Active Suicidal Ideation with | None during treatment | Fluoxetine 40 mg |
| | | Social | | | (Multi-year | Plan and intent | | Aripiprazole 2.5 mg |
| | | Phobia | | | history of tremor | 5 months prior to study entry | | Clonidine 0.1 mg |
| | | | | | prior to study) | Wish to be dead | | Clonazepam 0.5 mg |
| | | | | | | 2 weeks prior to study drug | | |
| 2 | 15 F | MDD | 76 | 44 (8) | Suicidal Ideation* | Suicide Attempt-Overdose | Active Suicidal Ideation | Fluoxetine 40 mg |
| | | Dysthymic | | | Sinus congestion* | 11 months prior to study entry | (1-2) | Ethinyl estradiol/ |
| | | Disorder | | | Dyspepsia* | Wish to be Dead | Resolved at week 3 | Levonorgestrel |
| | | Social | | | | 2 weeks prior to study drug | No recurrence | 20 mcg/0.1 mg |
| | | Phobia | | | | | | |
| 3 | 18 F | MDD | 59 | 29 (8) | Suicidal Ideation* | Suicide Attempt-Overdose | Wish to be Dead (1) | Fluoxetine 40 mg |
| | | Social | | | H1N1 influenza* | 5 years prior to study entry | Resolved at week 2 | Norgestimate/ |
| | | Phobia | | | Headache* | Wish to be Dead | No recurrence | Ethinyl estradiol |
| | | | | | Nausea/ | 1 week prior to study drug | | 0.25 mg/0.035 mg |
| | | | | | Vomiting* | | | |
| 4 | 14 F | MDD | 66 | 23 (8) | Suicidal Ideation* | Two Suicide Attempts- | Wish to be Dead (1-5) | Fluoxetine 20 mg |
| | | | | | Bruising* | Overdose | Resolved at week 6 | |
| | | | | | Headache* | 22 months prior to study entry | No recurrence | |
| | | | | | Acne* | 5 months prior to study entry | | |
| | | | | | URI* | Wish to be Dead | | |
| | | | | | | 1 week prior to study entry | | |
| 5 | 15 F | MDD | 62 | 33 (8) | URI* | No lifetime Suicidal Ideation | None during treatment | Fluoxetine 40 mg |
| | | Generalized | | | Epistaxis* | or Suicide Attempts | | |
| | | Anxiety | | | Headache* | | | |
| | | Disorder | | | | | | |

*Adverse Event Possibly or Probably Related to Study Drug
**Adverse Event Unrelated to Study Drug
CDRS-R: Children's Depression Rating Scale-Revised Raw Score
C-SSRS: Columbia Suicide Severity Rating Scale
MDD: Major Depressive Disorder
URI: Upper Respiratory Infection

3. ³¹P-MRS Neuroimaging

The depressed participants' ³¹P-MRS brain scans were performed at baseline (prior to the first dose of creatine) and repeated at the conclusion of treatment. We enrolled and acquired baseline scans from ten healthy female adolescent controls; six agreed to return eight weeks later for a second scan. Statistical analyses were performed using JMP 8 (SAS Inc., Cary, N.C. USA). Because this was an open-label pilot study, all analyses were considered exploratory and no correction for multiple hypothesis testing was applied.

A comparison of phosphorus metabolite levels between adolescent females with MDD and healthy controls is shown in Table 2 below. Our hypothesis that depressed participants would have a lower mean baseline b-NTP levels than controls was not confirmed (p=0.804; two-sample t-test, two-tailed). In fact, we found no statistically significant differences in PCr, pH or PCr/b-NTP ratio between adolescents with SSRI [symbol] resistant depression and controls.

TABLE 2
Comparison 31P-MRS Metabolites: Depressed
Adolescents vs. Healthy Controls.

| | Major Depressive | | |
|---|---|---|---|
| Phosphorus | Disorder | Healthy Controls | |
| Metabolite | (n = 5) | (n = 10) | p-value |
| PCr/TP | 0.1513 | 0.1526 | 0.853 |
| b-NTP/TP | 0.1254 | 0.1242 | 0.804 |
| PCr/b-NTP | 1.2217 | 1.2248 | 0.967 |
| pH | 7.0592 | 7.0439 | 0.567 |

Two sample t-test, 2-tailed
PCr: Phosphocreatine
beta-NTP: beta-Nucleoside Triphosphate
TP: Total Phosphorus Resonance
31P-MRS: ³¹-Phosphorus Magnetic Resonance Spectroscopy
MDD: Major Depressive Disorder

Table 3 below displays repeated measures of ³¹P-MRS metabolites in participants and controls, at baseline and 8 weeks later. Following 8 weeks of treatment with creatine, the depressed adolescents demonstrated a significant increase in mean PCr (p=0.020; paired t-test; 2-tailed). There was no change in creatine-treated participants' mean b [symbol] NTP, pH or PCr/b-NTP ratio.

TABLE 3
Baseline and Repeated 31P-MRS Results for Female
Adolescents with MDD and Healthy Controls.

| | Major Depressive Disorder | | | Healthy Controls | | |
|---|---|---|---|---|---|---|
| | (n = 5) | | | (n = 6) | | |
| | Mean | | | Mean | | |
| Phosphorus | Base- | Mean | p- | Base- | Mean | p- |
| Metabolite | line | 8 Weeks | value | line | 8 Weeks | value |
| PCr-TP | 0.1525 | 0.1610 | 0.020 | 0.1556 | 0.1558 | 0.969 |
| b-NTP/TP | 0.125 | 0.128 | 0.729 | 0.1203 | 0.1253 | 0.329 |
| pH | 7.059 | 7.071 | 0.648 | 7.0526 | 7.0294 | 0.054 |
| PCr/b-NTP | 1.221 | 1.270 | 0.528 | 1.2962 | 1.2584 | 0.645 |

Paired t-test, 2-tailed
PCr: Phosphocreatine
b-NTP: beta-Nucleoside Triphosphate
TP: Total Phosphorus Resonance
³¹P-MRS: ³¹-Phosphorus Magnetic Resonance Spectroscopy

Following the technique of a previous report (Renshaw et al. 2001), correlations between baseline depression rating scale scores and baseline metabolite levels were assessed using Spearman rank correlation and generalized two-tailed least squares modeling methods. CDRS-R baseline score was correlated with baseline pH (correlation=0.891 9; 95% CI 0.045-0.993; p=0.042). CDRS-R baseline score was negatively correlated with b-NTP concentration (Spearman's p=−0.90; p=0.037).

4. Adverse Events Associated with Creatine Treatment

At the fixed dose of 4 grams daily, creatine was well tolerated by study participants. Adverse events, which are summarized in Table 1 above, were self-limited: no participant experienced an unresolved medication-related side effect. None of the participants attempted suicide, engaged in self-injury or required psychiatric hospitalization during the study. There were no Serious Adverse Events (SAEs) (Food and Drug Administration 1996).

Participants' vital signs were recorded at each study visit. No statistically significant changes in weight, blood pressure or heart rate were found. Although not measured systematically, participant and parent acceptance of creatine as a treatment intervention appeared good, and there were no premature terminations due to concerns about the study drug. The laboratory tests performed at baseline and repeated after eight weeks of creatine revealed no clinically significant changes or abnormalities. No participant developed microalbuminuria or a serum creatinine outside the reference range in response to treatment with creatine.

Participants' suicidality was closely monitored using the C-SSRS; the results are summarized in Table 1 above. One participant reported no history of suicidal thoughts or behavior, but the others each endorsed significant histories of suicidality, indicating the severity and chronicity of the participants. Three of the participants had attempted suicide between 5 months and 5 years prior to study entry; at intake, four participants reported suicidal ideation in the 2 weeks prior to their baseline visit. During the treatment phase of the study, two participants reported no suicidality. Three participants reported suicidal ideation as treatment began, which lasted between 2 and 6 weeks. The participants' suicidal thoughts resolved during treatment with creatine and did not recur—either during the remainder of the 8-week treatment phase or at the 10-week follow-up visit. The participant who experienced suicidal ideation for the first 6 weeks of treatment had been burdened with chronic suicidality for the 22 months prior to study entry.

Overall, the study's drug-related adverse events were consistent with previous reports from our group and other investigators conducting studies of creatine in psychiatric disorders (Roitman et al. 2007; Kaptsan et al. 2007; Renshaw and Lyoo 2008; Lyoo et al. 2003).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of reducing likelihood of depression symptoms in a subject comprising the step of administering to the patient an effective amount of at least one creatine analog within ten days of administration to the subject an agent known to have a side effect of causing depression.

2. The method of claim 1, wherein the agent known to have a side effect of causing depression is selected from an anticonvulsant, a barbiturate, a benzodiazepine, a 13-adrenergic blocker, a calcium channel blocker, an estrogen, a fluoroquinolone, an interferon alpha, an opiod, and a statin.

3. The method of claim 1, wherein the agent known to have a side effect of causing depression is selected from Abilify® (aripiprazole), Accutane® (isotretinoin), Ambien® (zolpidem), Antabuse® (disulfuram), Chantix® (varenicline), Lariam® (mefloquine), Norplant® (levonorgestrel Implant), Parlodel® (bromocriptine), Savella® (milnacipran), Singulair® (montelukast), Strattera® (atomoxetine), tetrabenazine, tramadol, and Zovirax® (acyclovir).

4. A method for the treatment of a patient diagnosed with a depression disorder comprising the step of administering to the patient, together in a therapeutically effective amount, at least one creatine analog and at least one agent known to treat a depression disorder.

5. The method of claim 4, wherein the agent known to treat a depression disorder is selected from selective serotonin reuptake inhibitor, serotonin-norepinephrine reuptake inhibitors, tricyclic antidepressants, tetracyclic antidepressants, phenylpiperazine antidepressants, monoamine oxidase inhibitors, and atypical antidepressants.

6. The method of claim 5, wherein the agent known to treat a depression disorder is selected from fluoxetine, escitalopram, citalopram, paroxetine, fluvoxamine, and sertraline.

7. The method of claim 5, wherein the agent known to treat a depression disorder is selected from venlafaxine, desvenlafaxine, duloxetine, and milnacipran.

8. The method of claim 5, wherein the agent known to treat a depression disorder is selected from amoxapine, imipramine, trimipramine, nortriptyline, clomipramine, amitriptyline, doxepin, protriptyline, and desipramine.

9. The method of claim 5, wherein the agent known to treat a depression disorder is selected from tranylcypromine, isocarboxazid, selegiline, and phenelzine.

10. The method of claim 4, wherein the agent known to treat a depression disorder is a selective serotonin reuptake inhibitor.

11. A method for the treatment of a depression disorder in a selective serotonin reuptake inhibitor-treatment resistant patient comprising the step of administering to the patient an effective amount of a selective serotonin reuptake inhibitor and an effective amount of at least one creatine analog.

12. The method of claim 11, wherein the selective serotonin reuptake inhibitor is selected from fluoxetine, escitalopram, citalopram, paroxetine, fluvoxamine, sertraline, venlafaxine, desvenlafaxine, duloxetine, and milnacipran.

13. A method for the treatment of a subject comprising the steps of:
a) diagnosing the subject as having a depression disorder; and
b) administering to the subject an effective amount of at least one creatine analog.

14. The method of claim 13, wherein diagnosing comprises performing a ³¹P MRS experiment upon the subject and identifying a level of a metabolic marker.

15. The method of claim 14, wherein a metabolic marker comprises at least one of magnesium, pH, total nucleoside concentration, phosphocreatine, and β nucleoside triphosphate.

16. The method of claim 13, wherein diagnosing comprises the use of one or more of Adolescent Inventory of Suicide Orientation-30 (ISO-30), Adult Suicidal Ideation Questionnaire (ASIQ), Beck Hopelessness Scale (BHS), Beck Scale for Suicide Ideation (BSS), Child Suicide Risk Assessment (CSRA), Child-Adolescent Suicidal Potential Index (CASPI), Columbia Classification Algorithm of Suicide Assessment (C-CASA), Columbia Suicide Severity Rating Scale (C-SSRS), Coping Inventory for Stressful Situations (CISS), Firestone Assessment of Self-Destructive Thoughts (FAST), Lazurus' BASIC ID tool, Lifetime Parasuicide Count (LPC), MAST—Attraction to Death (MAST-AD), MAST—Repulsion by Life (MAST-RL), Modified SAD PERSONS Scale of Hockberger and Rothstein, Multi-Attitude Suicide Tendency Scale (MAST), Parasuicide History Interview (PHI), Positive and Negative Suicide Ideation Inventory (PANSI), Reasons for Living Inventory (RFL; either long form or short form), Reasons for Living Inventory for Adolescents (RFL-A), Reasons for Living Inventory for Young Adults (RFL-YA), Risk Factors of Powell et al for Predicting the Risk of Suicide in a Psychiatric Ward Inpatient, Risk-Rescue Rating (of Weisman and Worden for Suicide Assessment), Scale for Suicidal Ideation (SSI), Suicidal Behavior History Form (SBHF), Suicidal Behavior Questionnaire for Children (SBQ-C), Suicidal Ideation Questionnaire (SIQ), Suicidal Tendencies Test, Suicide Behaviors Questionnaire (SBQ), Suicide Behaviors Questionnaire-Revised (SBQ-R), Suicide Probability Scale (SPS), and Suicide Resilience Inventory-25 (SRI-25).

17. The method of claim 4, wherein the patient is an adolescent.

18. The method of claim 4, wherein the patient is female.

19. The method of claim 1, wherein the depression disorder is selected from major depressive disorder, minor depression disorder, dysthymia, postpartum depression, seasonal affective disorder, bipolar disorder, and suicidal ideation.

20. The method of claim 1, wherein the creatine analog comprises one or more of creatine, creatine monohydrate, creatine ethyl ester, creatine citrate, creatine malate, creatine tartrate, and magnesium creatine chelate.

21-22. (canceled)
